(19) United States
(12) Patent Application Publication
(10) Pub. No.: US 2006/0039954 A1
(43) Pub. Date: Feb. 23, 2006

(54) Ocular formulations with neuroprotectants to reduce Alzheimer and neurotoxic risks created by large zinc dosages

(76) Inventors: Dennis L. Gierhart, Chesterfield, MO (US); C. Kathleen Dorey, Boca Raton, FL (US); Patrick D. Kelly, Kirkwood, MO (US)

Correspondence Address: Patrick D. Kelly, #403, 11939 Manchester, St. Louis, MO, 63131, US

(21) Appl. No.: 11/133,125

(22) Filed: May 18, 2005

Related U.S. Application Data
(63) Continuation-in-part of application No. 10/746,403, filed on Dec. 23, 2003.
(60) Provisional application No. 60/572,214, filed on May 18, 2004.

(51) Int. Cl. A61K 47/00; A61K 33/32; A61K 31/375; A61K 31/355; A61K 31/12

(52) U.S. Cl. 424/439; 424/641; 514/690; 514/458; 514/474

(57) ABSTRACT

Formulations marketed to elderly consumers for preventing or treating age-related eye diseases such as macular degeneration are modified in various four ways, compared to the formulations tested in the first “Age-Related Eye Disease Study” (AREDS-1) clinical trial. Zinc dosages are substabtially reduced, to reduce the risk of Alzheimer's disease and other neurotoxic damage in the brains of elderly people, and zeaxanthin is substituted for a substantial portion of any beta-carotene. Additional useful agents are also disclosed.

### RELATED APPLICATIONS

This application claims the benefit under 35 USC 119(e) of provisional application 60/572,214, filed May 18, 2004. It also is a continuation-in-part of utility application Ser. No. 10/746,403, filed Dec. 23, 2003.

### FIELD OF THE INVENTION

This invention is in the fields of pharmaceuticals and nutritional supplements, and relates to orally-ingested formulations for treating or preventing macular degeneration and other eye and vision disorders.

### BACKGROUND OF THE INVENTION

The retina is the layer of nerve cells at the back of the eye, which convert light into nerve signals that are sent to the brain. In humans and other primates (but not in most other mammals), the macula is a small yellowish circular area in the retina, positioned at the center of the field of vision. It provides fine-resolution vision in the center of the visual field, and it is essential to good vision. People who suffer from macular degeneration often lose the ability to read, recognize faces, drive, or walk safely on unfamiliar routes.

Macular degeneration is the leading cause of severe vision loss and functional blindness among the elderly, and its rates are increasing as the population ages, and as dietary patterns shift away from dark green vegetables toward more fatty foods. The disease, and efforts to prevent or treat it, are described in many books (e.g., D'Amato et al 2000 and Lim 2002) and articles (reviews include Byrne et al 2003, Blodi 2004, and Zarbin 2004).

Since October 2001, scientific and medical reports and practices concerning macular degeneration have been dominated by two articles that were published back-to-back. Those articles reported data and conclusions from a very large clinical trial, discussed below. U.S. Pat. No. 6,660,297 (Bartels et al 2003, assigned to Bausch and Lomb) also is directly relevant, since it claimed certain dietary supplements tested in that study. The product being sold by Bausch and Lomb under that patent is called OCUVITE PRESERVISION™. It contains a very heavy dosage of zinc, and it is labelled and advertised in ways that recommend ingestion by elderly people, each and every day. A nearly identical formulation called ICAPS AREDS™ is also sold by Alcon Laboratories; those two companies initially engaged in litigation over U.S. Pat. No. 6,660,297, but the lawsuit was settled under terms that allow both companies to continue selling their AREDS products.

The AREDS-1 Study: Anti-Oxidants and Zinc

In the 1990's, the National Eye Institute (unless otherwise noted, any agencies referred to herein are in the United States) organized and carried out a major clinical trial involving thousands of patients who were suffering from (or at high risk of) macular degeneration and/or cataracts. The trial was partially sponsored by Bausch & Lomb, but it also received tens of millions of dollars in taxpayer funding.

Macular degeneration and cataracts are clearly related to aging. They strongly increase in frequency and severity as people pass beyond the age of about 60, and they rarely if ever occur in anyone under the age of about 50, except in people who have specific genetic defects in their eyes. Accordingly, the trial was called the “Age-Related Eye Disease Study”, abbreviated as AREDS. As this is being written, proposals are being discussed for a followup study that likely will be called the AREDS-2 study. Therefore, the study carried out in the 1990's is referred to herein as AREDS-1.

AREDS-1 tested a combination of vitamins that share an important trait: they all have anti-oxidant activity. This trait refers to their ability to neutralize (“quench”) certain types of destructive molecules usually referred to as reactive oxygen species, or as oxygen (or oxidative) free radicals. These compounds also are referred to as “free radicals” or “radicals”, for convenience.

These radicals contain oxygen atoms with an unpaired electron. They are highly unstable and aggressively reactive, and they will attack nearly any type of biological molecule. In humans, oxygen radicals cause three major types of damage: (1) alterations in DNA strands, which can lead to permanent mutations that will be inherited by other cells, including mutations that can increase the risks of cancer and other diseases; (2) alterations in proteins, which can render enzymes or structural proteins defective and unable to function; and, (3) damage to cell membranes.

These types of destructive radicals frequently are created when radiation in the deep blue and ultraviolet (UV) regions of the spectrum hits biological molecules. Deep blue and UV radiation carry higher levels of energy than photons with longer wavelengths, and such high-energy radiation can randomly break apart most types of biomolecules. Over a long lifespan, UV and deep blue light will enter the eyes of any person in large quantities, on a cumulative basis. The damage they cause are presumed to be contributing factors whenever the eyes of an elderly person begin suffering from a disorder that is known to be age-related, such as macular degeneration or cataracts.

As suggested by their names, “anti-oxidants” are molecules that are effective and useful in neutralizing and “quenching” oxidative radicals having unshared electrons. Different anti-oxidants achieve that result by different mechanisms. In some cases, an anti-oxidant will donate an electron that it can spare, to stabilize a radical with a “singlet” electron that needs another electron to become stable. In other cases, an anti-oxidant can take away one electron from a radical that needs to get rid of a “triplet” electron to become stable. In still other cases, an anti-oxidant can bond to a free radical in a manner that creates a stabilized molecular complex that contains both the radical and the anti-oxidant.

These types of reactions involve complex electron chemistry, and they can be carried out effectively by only a few types of special compounds that are stable and non-harmful, in animal or plant cells and tissues. However, radical-stabilizing reactions are well-known to experts, and several biological compounds are known to be effective anti-oxidants.

Vitamins C and E are at the top of nearly any list of healthy and useful anti-oxidants, for humans. Both have been recognized as vitamins for decades; they are essential for growth and health, they are readily available at low cost, and their anti-oxidant activity is well-known and well-understood. Vitamin C is the common name for ascorbic acid, and also is referred to by terms such as ascorbate (which refers to the ionized form of the acid, or to a stable salt) or ester forms, such as ascorbyl palmitate (which refers to a stable precursor that is cleaved in the body, to release the vitamin). Vitamin E is the common name for a compound called alpha-tocopherol; however, since a number of other closely-similar tocopherol compounds also have good anti-oxidant activity, many scientists and specialists often use the term “tocopherols” interchangeably with Vitamin E.

Vitamins C and E were included in the AREDS-1 trial because they are undoubtedly beneficial, especially among many seniors, who do not always eat balanced diets (especially those who are on fixed incomes and/or are struggling to pay high medical and drug bills). They were tested in large dosages, based on decisions made in the early 1990's, when the AREDS-1 trial was being planned and organized. However, in recent years, a number of reports have indicated that very high dosages of Vitamin E may cause adverse effects (such as cardiovascular problems) in some segments of the population.

Beta (B) carotene, which is cleaved in half to release two molecules of Vitamin A, also is a well-known anti-oxidant. It belongs in a class of compounds called carotenoids, discussed below. It was included in the AREDS-1 trial, at very high dosages; however, as with Vitamin E, recent reports have indicated that it raises major concerns and risks, when very high dosages are consumed. Accordingly, any discussion of β-carotene, in possible treatments for macular degeneration or cataracts, must take into account the following factors:
1. In recent years, scientists have realized that β-carotene is not deposited in the macula (the yellow-colored spot that is crucial for good vision, at the center of the retina), in human eyes. Therefore, a consensus has emerged among eye and vision researchers that high dosages of β-carotene do not offer any realistic promise of providing any substantial benefits or protection against macular degeneration, among people who receive baseline levels;
2. Published scientific reports (e.g., Burton et al 1984) indicate that β-carotene actually reverses its anti-oxidant activity, and becomes a pro-oxidant, if unusually high concentrations of oxygen are present. These oxygen concentrations are not present in most tissues and fluids in the body; however, since the lungs interact directly with oxygen in air that is breathed, β-carotene may act as a damaging pro-oxidant, rather than a beneficial anti-oxidant, in lung tissues;
3. As an apparent result of its damaging pro-oxidant activity at high oxygen concentrations, large and well-run clinical trials have convincingly shown that high-dosage β-carotene, instead of being useful and protective, actually increases the risks of lung cancer among smokers. This clearly and unmistakably occurs among smokers, and it may also be happening to a lesser extent among non-smokers.

As a result, some experts have stated, openly and in print, that they believe high-dosage β-carotene supplements are unsafe and should be taken completely off the market, and β-carotene should not be given to anyone except at low dosage levels as used in common multi-vitamins.

Accordingly, the Bartels et al '297 patent (which was filed in 1999, after researchers had begun to realize that β-carotene probably was not helping protect the macula) specifically stated that β-carotene could be deleted from the anti-oxidant combination that was tested in the AREDS-1 trial, and it could be replaced by either or both of two other carotenoids, lutein and zeaxanthin, which are discussed below.

In addition to the triple-antioxidant combination that contained Vitamin C, Vitamin E, and β-carotene (the precursor of Vitamin A), some of the populations of elderly people who were tested in the AREDS-1 study also received zinc. This was done because a number of earlier reports had stated that oral zinc supplements appeared to help some macular degeneration sufferers. Those reports include Newsome et al 1988, Yuzbasiyan et al 1989, Hawkins 1991, Trempe 1992, and Beaumont 1993. Accordingly, the four “arms” of the AREDS-1 study were antioxidants alone, antioxidants plus zinc, zinc alone, or nothing (controls).

Zinc oxide was chosen as the zinc compound used in AREDS-1. Dosages used in the trial are believed to be 80 mg/day of zinc oxide; however, the published AREDS reports refer to “zinc, 80 mg, as zinc oxide”. That raises questions as to whether the “80 mg” dosage refers to elemental zinc (molecular weight 65.4), or zinc oxide (molecular weight 81.4).

Daily zinc dosages in OCUVITE PRESERVISION (Bausch and Lomb, B&L) clearly are 69.6 mg of elemental zinc, as zinc oxide. That 69.6 mg/day dosage of elemental zinc is claimed and advertised by B&L as equal to 464% of the “Recommended Daily Allowance” (RDA) for zinc; however, the 464% number is based on an old and outdated RDA value of 15 mg/day. Current RDA values, published in 2001 by the Food and Nutrition Board (part of the Institute of Medicine) states that the RDA for elemental zinc is 8 milligrams per day for adult females, and 11 milligrams per day for adult males. Therefore, a daily dosage of 69.6 mg of elemental zinc, from OCUVITE PRESERVISION pills alone, without considering other dietary sources, is actually 870% of the RDA for women, and 633% of the RDA for men, rather than the 464% number claimed and advertised by B&L.

Elemental zinc dosages in ICAPS AREDS™, sold by Alcon, are also 69.6 mg/day; however, Alcon's product contains zinc acetate, a salt that readily releases zinc ions in aqueous liquids, while zinc oxide is not a salt and has low solubility in water.

It also should be noted that Storz Ophthalmics, a company purchased in B&L in the mid-1990's, was the originator of the OCUVITE product. An OCUVITE product sold by Storz in 1993 (which grew out of Newsome's 1998 report) contained zinc at 40 mg, copper at 2 mg, Vitamin C at 60 mg, Vitamin E at 30 IU, beta-carotene at 5000 IU, and selenium at 40 micrograms, all as daily dosages.

In addition to “Recommended Daily Allowance” (RDA) numbers, the Institute of Medicine has recently adopted and issued “Tolerable Upper Intake Levels” for various nutrients. For zinc, “Tolerable Upper Intake Levels” were set in 2001 at 40 mg/day for both men and women. Therefore, the amount of zinc in OCUVITE PRESERVISION, by itself, appears to be nearly twice as high as the “Tolerable Upper Intake Levels” set by the Institute of Medicine, and the surplus becomes even higher if additional zinc intake in the diet is also included. RDA and “Tolerable Upper Intake Levels” are discussed in more detail below.

Returning to the AREDS-1 trial, the design and results of the study were complicated, and involved (i) multiple subdivided test populations, and (ii) treatment classifications that were changed, after the data had been analyzed. As mentioned above, participants were divided into four treatment arms, which were antioxidants alone, antioxidants plus zinc, zinc alone, or nothing (controls). In each treatment arm, participants were divided into four categories, depending on their eye health when they entered the study. Category 1, at the low end of the scale, contained people with no apparent signs of macular problems. Category 4, at the high end of the scale, contained people with serious problems in one eye while the other eye remained sufficiently free of advanced problems to allow monitoring and measuring of subsequent declines, after the person began taking supplements.

The main results of AREDS-1 were published in two articles in the October 2001 issue of Archives of Ophthalmology (published by the American Medical Association). Macular degeneration data were reported in AREDS Report Number 8 (Arch. Ophthalmol. 119: 1417-1436). Cataract data were reported in AREDS Report Number 9 (Arch. Ophthalmol. 119: 1439-1452). Both reports appeared under the byline, “Age-Related Eye Disease Study Research Group”, with members listed at Arch. Ophthalmol. 119: 1437-1438.

While AREDS Report 8 should be consulted for details, its results generally can be summarized as follows:
(1) Test patients who suffered from moderate or advanced macular degeneration, and who received Vitamins A, C, and E but no zinc, showed some positive results, but the indicators did not rise to a level of statistical significance;
(2) Test patients who suffered from moderate or advanced macular degeneration, and who received zinc but no antioxidant vitamins, also showed some positive results, but the indicators did not reach statistical significance;
(3) A third set of test patients who suffered from moderate or advanced macular degeneration, and who received both zinc and antioxidants, showed positive results that reached a level of statistical significance.

Those results can be interpreted either positively or negatively. Various commentators described the findings favorably, and asserted that the combination of β-carotene, Vitamins C and E, and zinc were the first treatment ever proven to actually slow down macular degeneration. However, others asserted that the levels of protection were disappointingly weak, and benefits were shown only in certain limited classes of patients. A sharply critical scientific analysis was published by Siegel 2002, entitled, “AREDS Investigators Distort Findings,” which asserted that any claimed benefits of the combined antioxidant-zinc treatment could be teased out of the data only by massaging the data in ways that, instead of being objective and impartial, were biased and intended to locate something positive to report, to offset the fact that the entire remainder of the study had spent many millions of dollars but had come up empty. In the words of that expert, “In my opinion the AREDS investigators promoted a nonsignificant result into a conclusive recommendation. Here is how they did it. . . . the message that should have emerged from AREDS is that these treatments failed to demonstrate efficacy in preventing AMD and are not recommended for that use.”

Even the reviewer who mostly endorsed the AREDS findings, in an editorial that accompanied AREDS Reports Numbers 8 and 9 in the same issue of the same journal, had to include statements such as, “The exclusion of the subgroup of patients in Category 2 from many of the analyses because of the low incidence of primary outcome events is troubling because it came after review of the data” (Jampol 2001, Arch. Ophthalmol. 119: 1534). Two years later, that same reviewer published an update containing statements such as, “The potential toxicity of both the AREDS formulation and these other preparations should be kept in mind. . . . Evidence supports the use of the AREDS supplements only in patients with intermediate or advanced AMD” (Jampol 2003).

Jampol 2003 also pointed out a number of growing concerns over the very high dosages of the other agents in the AREDS-1 formulations. For example, Vitamin A and its precursor, beta-carotene, are associated with decreased bone density, and elevated risks of bone fractures. Clearly, those bone-related risks are of major concern, among the elderly.

In addition, even the safety of high dosages of Vitamins C and E have been called into question. For example, studies have indicated that post-menopausal women who suffer from coronary artery disease should not take high dosages of vitamins C or E. At first glance, that might seem like a relatively small subcategory of people who should be disqualified from taking AREDS supplements; however, it actually is a large and important subset of people who are concerned about macular degeneration. Age-related macular degeneration doesn't become a significant concern until people reach their 60's, 70's, and 80's; therefore, nearly any woman who must decide whether to take an AREDS supplement will indeed be of post-menopausal age. After that factor has been recognized, it must then be recognized that nearly everyone who has reached that age has some level of plaque buildup in their arteries, especially if they live in an industrialized country where food intake over the course of a lifetime includes substantial quantities of fatty foods (this factor deserves attention, since fatty foods simultaneously increase both the risks of arterial plaques, and the risks of macular degeneration, thereby creating a causative link and overlap between those two problems). Therefore, serious questions must be asked about whether, and under what conditions, elderly women (especially elderly women who may be a few pounds overweight, and/or who have high cholesterol levels) can safely take heavy dosages of vitamins C and E. These and similar concerns about widespread, uncontrolled, and potentially dangerous intake of high-dosage vitamins are discussed in items such as Gaynes 2003 and Pulido et al 2004 (which relate specifically to the AREDS products that currently are being sold to elderly people), and in numerous other articles that discuss general concerns about heavy vitamin intake (often referred to as “mega-vitamin” intake).

This current invention focuses mainly on yet another problem that apparently was never recognized or addressed in U.S. Pat. No. 6,660,297 (Bartels et al), or in any of the written papers that were submitted to the Patent Office by the inventors of that patent, during the prosecution of that patent. With a few limited exceptions, described below, this problem also was never recognized or addressed by any of the members of the panel of experts Who designed, managed, and published the results of the AREDS-1 trial.

The problem addressed by this invention relates to concerns that extra-heavy dosages of zinc may cause serious risks, not among all elderly consumers, but among sufficient numbers to create major concerns. Three concerns involve neurology; one focuses on Alzheimer's disease and beta-amyloid plaques, one focuses on the severity of brain damage and permanent impairment following a stroke, cardiac arrest, or other crisis that assaults the brain, and one focuses on cognitive impairments seen in animals that were fed heavy dosages of zinc.

Another non-neurological risks that needs attention is the risk that heavy zinc intake may trigger or stimulate the growth of prostate cancer, among middle-aged and elderly men. In addition, zinc has been discovered in high concentrations in unwanted deposits called “drusen”, in human retinas. This raises concerns that heavy zinc intake may accelerate the formation and growth of those unwanted deposits, which can disrupt the retina and damage vision.

These risks are described in more detail below.

Zinc in Beta-Amyloid Plaques and Alzheimer's Disease

A number of published reports, which initially were criticized but which recently have gained strongly in their level of credibility and acceptance among neurologic researchers, have reported a strong link between zinc levels in the brain, and Alzheimer's disease.

This link focuses on β-amyloid plaques, one of the primary and even defining traits of Alzheimer's disease. Reviews of these plaques include Turner et al 2003, Chaney et al 2003, and Kar et al 2004, and they can be briefly summarized as follows.

The β-amyloid protein is a relatively short polypeptide that is cleaved off from a larger polypeptide called the “amyloid precursor” protein or polypeptide (abbreviated as APP). The complete APP protein straddles a cell membrane, with a small intra-cellular portion extending into the fluid inside a cell (i.e., the cytoplasm), and a larger extra-cellular portion exposed to blood, lymph, and other fluids outside the cell.

Various segments are cleaved off of the extra-cellular portion of the APP protein, by enzymes. A relatively large fragment that is cleaved off is called apolipoprotein E (often abbreviated as apoE). When apoE is cleaved from the extra-cellular portion of the amyloid precursor protein, it becomes a soluble blood protein that helps transport cholesterol to tissues in a natural and necessary manner.

The β-amyloid protein, which is heavily involved in Alzheimer's disease, is a smaller fragment. It apparently can be created by either of two processes: (i) cleavage from the extracellular domain of the cell-bound APP protein, or (ii) cleavage from soluble apolipoprotein E fragments that have already been separated from the cell-bound APP protein.

Everyone has the apoE protein fragment in their blood. Everyone also has the smaller β-amyloid fragment in their blood, in soluble form, at low concentrations. However, in victims of Alzheimer's disease, the β-amyloid fragment begins to accumulate in clumps, inside brain tissue. Some of these clumps are wedged in between cells called “glial” cells, which includes cells other than neurons that are present in brain and spinal tissue. Unlike neurons, glial cells cannot receive or transmit nerve impulses, and their role is to help support, nurture, and protect the neurons.

Other β-amyloid plaques are packed tightly around capillaries inside the brain. Those plaques are suspected, by at least some researchers, of being a response to problems that arise when capillaries in the brain become “leaky” and can no longer maintain the so-called “blood brain barrier” (BBB). The BBB is formed by “tight junctions” between the overlapping endothelial cells that form capillaries, inside the brain and spinal cord. Those “tight junctions” make the walls of capillaries in the brain and spinal cord much less permeable than the walls of capillaries elsewhere in the body. This helps protect the brain against molecules that would interfere with proper functioning of neurons in the brain and spinal cord.

In elderly people, the tight-junctioned capillary walls in the brain sometimes begin to leak small quantities of blood into surrounding tissue. This is a known problem, and at least some researchers suspect that, in at least some patients, β-amyloid plaques are a response to that problem. Those plaques, when found sticking to capillary walls, are analogous to “plumber's putty”, the sticky waterproof compound that plumbers sometimes use to slow down or stop some types of slow leaks that cannot be fixed by tightening a fitting.

Scientific and medical opinions are divided over whether β-amyloid plaques are one of the causes, or one of the results, of Alzheimer's disease. However, those plaques clearly and unmistakably can be found, after autopsy, in the brain of anyone who suffered from Alzheimer's disease when he or she died. Indeed, β-amyloid plaques generally are regarded as a defining trait of Alzheimer's disease, and their presence is used to distinguish Alzheimer's disease from other forms of dementia and memory loss among aged people.

The formation and growth of β-amyloid plaques, in the brain of someone suffering from Alzheimer's disease, is a major and crucial departure from the presence of low quantities of soluble β-amyloid protein in the circulating blood of a healthy person. When β-amyloid plaques begin forming in significant quantities inside the brain, something has gone seriously wrong, and the pathological process of Alzheimer's disease has commenced.

As mentioned above, a number of published articles have clearly established that zinc is present in unusually high quantities, in β-amyloid plaques in people with Alzheimer's disease. Copper is also present, to a lesser extent; however, since zinc is present at much higher concentrations than copper throughout the body, the primary focus of this line of research has been on zinc.

Zinc is present in every type of tissue in the body; indeed, because of “zinc finger” proteins, which are essential in enzymes that interact with DNA, zinc is present in every living cell on earth. It is present in all mammalian brains; it is even released by some types of neurons, when they transmit nerve impulses. Accordingly, a substantial amount of zinc, in the brain, is absolutely essential in all humans and other animals.

However, when people begin to suffer from Alzheimer's disease, something goes wrong in the way the brain is handling and processing zinc. This malfunction is sometimes referred to as “zinc dyshomeostasis”, which needs to be understood.

“Homeostasis” is a crucial biological process, discussed in any textbook on physiology (e.g., page 4 of the Textbook of Medical Physiology by Guyton and Hall, 9th edition, 1996). It refers to the maintenance of reasonably consistent and properly functional conditions, within a “steady-state” set of boundaries and constraints, in an organism. This does not mean the processes inside an organism remain static and unchanging, because nothing in life or biology is static and unchanging. Instead, it implies and requires that: (i) all essential conditions (such as body temperature, blood flow rates, etc.) must remain within a set of reasonable and proper boundaries and constraints; and, (ii) when a disruption occurs, the organism and its components (including its internal organs, muscles, nervous system, etc.) will respond to the disruption in ways that help bring the organism back toward a stable ongoing equilibrium.

In most people, the body and brain are capable of sustaining homeostasis for well over 70 years. However, in people suffering from infections, diseases or other serious disorders, poor diets, lack of exercise, long-term smoking, etc., conditions often occur that can be called “dys-homeostasis”, where the negative prefix “dys-” indicates that something has gone wrong.

Accordingly, the term, “zinc dyshomeostasis”, when applied to a person suffering from Alzheimer's disease, indicates that something has gone wrong with that particular person's ability to process and metabolize zinc, inside his or her brain. It doesn't happen to everyone, as evidenced by the fact that not everyone gets Alzheimer's disease. However, as evidenced by the rates of occurrence of Alzheimer's disease (these rates are increasing substantially and even alarmingly, as the population ages, and as medical advances enable more people to live past the age of 80), it happens often enough to be a hugely important and horribly expensive problem, in society and medicine.

Under the “plumber's putty” hypothesis, which suggests that β-amyloid plaques are being used to help prevent blood from leaking out of capillary walls in the brain that are becoming leaky, it should not be surprising that zinc would be present in relatively high quantities in such plaques. A single ion of zinc can form cross-linking bonds with up to four different amino acid residues in proteins. Since a zinc ion is positively charged (Zn⁺⁺), it forms cross-linking bonds with negatively-charged unshared electron pairs on the side chains of two particular types of amino acids, cysteine and histidine. This allows an ion of zinc to hold together different segments or strands of protein. As an example, in most zinc finger proteins, a single zinc ion sits at the base of each “finger” domain, with a total of four cysteine or histidine residues cross-linked to the zinc ion.

The cross-linking bonds formed between zinc ions and cysteine or histidine residues in proteins are not covalent. Nevertheless, they are relatively strong and stable, and in most proteins, they generally last for the life of the protein. Therefore, cross-linking bonds involving zinc and proteins are sometimes called “coordinate” or “metastable” bonds, which implies that they are stronger than ionic bonds, even though they are not in the strongest category of covalent bonds.

For years, the presence of high levels of zinc in β-amyloid plaques was either ignored, or regarded merely as a byproduct of the plaque formation process. However, in 1994, Dr. Ashley Bush, at Harvard Medical School, coauthored a paper with several colleagues, published in Science, one of the most prestigious scientific journals in the world. That paper (Bush et al 1994, entitled, “Rapid induction of Alzheimer beta amyloid formation by zinc”) set forth a theory that a buildup of zinc, in the brains of people suffering from Alzheimer's disease, might be provoking or aggravating the formation and growth of β-amyloid plaques.

That hypothesis was harshly criticized by other neurology and Alzheimer researchers, as described in an article on page A1 of the Wall Street Journal on Dec. 26, 2003 (Wysocki 2003). However, as described in that same article, Dr. Bush persisted in gathering data and exploring his hypothesis, and he located an old drug called clioquinol, which was used in the 1940's to treat dysentery. Clioquinol is a zinc “chelator”, which means it will bind to zinc in a way that inactivates and sequesters the zinc. Chelation is analogous to placing a wrapper around a piece of sticky candy; as long as the candy is stuck to the wrapper, it cannot stick to anything else.

The medical use of clioquinol was terminated in the late 1940's, because it caused severe side effects (including strokes and paralysis) in some patients. However, Dr. Bush studied those side effects, and became convinced they were being caused or aggravated by deficiencies of vitamin B12, rather than by the clioquinol, since the toxic side effects arose mainly in Japan during the years after World War Two, when vitamin deficiencies were endemic and severe, as much of Japan suffered from borderline starvation while it struggled to recover from the destruction of the war.

Dr. Bush and his coworkers began doing animal studies, to test whether clioquinol (supplemented by Vitamin B12, to avoid the risk of toxic side effects) could prevent β-amyloid plaque formation in genetically-engineered mice that rapidly accumulate β-amyloid plaques. The results were positive, as described in Cherny et al 2001. Those results from animal tests led to human clinical trials, which also led to positive and promising results, as described in Ritchie et al 2003. These developments are described in more detail in various articles such as Bush 2003 and Finefrock et al 2003.

In addition, a link has been reported between Alzheimer's disease, and elevated levels of zinc in blood serum. As reported in Rulon et al 2000, based on post-mortem studies of nine Alzheimer patients and eight age-matched non-Alzheimer controls, a statistically significant elevation of zinc serum was observed in the Alzheimer patients (136.4±66.8 micrograms per deciliter, or μg/dL), compared to controls having only about half that level (71.1±35.0 μg/dL).

Other articles shed more light on the subject, and several articles (including Kagan et al 2002, Hooper et al 2002, Allinson et al 2003, and Cuajungco et al 2003) describe factors that contribute to what Cuajungco et al call “The Zinc Paradox”. This paradox refers to the fact that zinc appears to play two different and contradictory roles, in Alzheimer's disease. Initially, it apparently stimulates the formation and growth of β-amyloid plaques, as discussed above. However, after β-amyloid plaques have been formed, zinc apparently attempts to help reduce and minimize the damage caused by those plaques. This type of “damage-control” activity apparently involves at least two factors: (i) the quenching of destructive oxygen free radicals, which are generated in and around the plaques by pathways involving hydrogen peroxide, and (ii) the blockage, by zinc ions, of uncontrolled ion flow into and out of neurons, via nonselective ion channels.

Readers should also note that zinc is involved in another important class of enzymes, called alpha-secretase enzymes. These enzymes apparently can help promote the processing of amyloid precursor protein (APP) in useful ways, which may help prevent the APP protein from being mishandled in ways that will create or aggravate β-amyloid plaques (e.g., Hooper et al 2002).

Despite the complexities involved, it is clear that in some portion of the elderly population, a potentially pathological problem that has been called “zinc dyshomeostasis” appears to trigger and/or aggravate the formation and growth of β-amyloid plaques, which are directly and heavily involved in Alzheimer's disease.

That leads to a problem that can be stated as follows: under the current state of medical knowledge, out of the millions of elderly people who will consume a supplement that reportedly helps reduce the risk or severity of macular degeneration, no one can know which particular elderly consumers will suffer from the type of “zinc dyshomeostasis” that may trigger or accelerate Alzheimer's disease, if they begin swallowing dosages of zinc that are nearly twice as high as the “tolerable upper limits” recommended by the top nutrition experts at the Institute of Medicine.

Since no one can know or predict which particular elderly consumers might be triggered or nudged toward Alzheimer's disease by extra-heavy dosages of zinc interacting with a pre-existing “zinc dyshomeostasis” problem, serious questions need to be asked, promptly and even urgently, about the medical safety of eye nutrition products that contain extra-heavy dosages of zinc, especially if those products are being specifically and deliberately advertised and marketed to elderly people.

The OCUVITE PRESERVISION and I-CAPS AREDS products, for preventing or treating macular degeneration, are indeed being advertised and marketed, in a deliberately targeted manner, to elderly people. Macular degeneration is clearly and undeniably a disease that is limited almost exclusively to the elderly; except for people with specific genetic defects that damage their eyes, macular degeneration occurs almost exclusively among people more than 60 years old (indeed, most patients are over the age of 70).

Accordingly, this invention discloses improved formulations that can provide the benefits of zinc supplements in preventing or treating macular degeneration, while also minimizing the risks that extra-heavy dosages of zinc might accelerate the creation and/or growth of β-amyloid plaques, and the onset and/or severity of Alzheimer's disease, in at least some elderly consumers.

Before describing how that goal can be accomplished, it must also be noted that extra-heavy zinc dosages in nutritional products targeted at elderly consumers also pose another important neurological threat, described below.

Links Between Elevated Zinc and Other Neurotoxicity

In addition to the concerns about Alzheimer's disease, a second and completely different set of research reports raises major concerns about whether extra-heavy dosages of zinc, if taken on a daily basis by elderly people, will create or aggravate another type of neurotoxic damage. These concerns focus on increased severity of permanent brain damage and impairment, if a stroke, cardiac arrest, head injury, or other medical crisis that affects the brain occurs in someone who has been taking unusually heavy dosages of zinc.

These concerns are described in numerous articles published in respected science and medical journals, by numerous research teams. A search of the National Library of Medicine database (www.ncbi.nlm.nih.gov/pubmed) combining words such as “zinc” and “neurotoxic” will quickly generate a listing of titles and abstracts such as quoted below. Alternately, a search of “Choi” and “zinc” will reveal numerous articles coauthored by a prominent researcher in this field, Prof. Dennis Choi at the Washington University School of Medicine, in St. Louis.

These articles do not assert that zinc will be neurotoxic in all elderly patients. Instead, these articles recognize and confirm that zinc is present in all mammalian brains, and that zinc is normally and naturally released by neurons, as an essential part of nerve signal transmissions. Nevertheless, these articles consistently state that elevated concentrations of zinc can aggravate and increase neuronal damage and death, if a crisis occurs that involves ischemia and/or hypoxia.

“Ischemia” refers to inadequate blood flow. It occurs inside a brain when a person suffers a stroke, burst aneurysm or other hemorrhage, etc. It also occurs when a person suffers a cardiac arrest (i.e., when the heart stops beating, during a major heart attack), or severe blood loss due to an injury, trauma, major surgery, etc. It also occurs when a person suffers a head injury that causes brain swelling, since elevated fluid pressures on capillary walls in the brain will tend to flatten and constrict the very thin flexible walls of the capillaries.

“Hypoxia” refers to inadequate oxygen supply. It occurs as a direct result whenever blood supply is hindered, during a stroke, cardiac arrest, or other ischemic crisis as listed above. It also occurs during other conditions when oxygen supply is reduced, such as during a near-drowning, suffocation, or asphyxiation, or in carbon monoxide poisoning.

Strokes, cardiac arrest, and other ischemic and hypoxic crises are regrettably frequent and common, among the very same elderly patients who are worried about losing their eyesight due to macular degeneration. Therefore, the statements quoted below, about the ways in which zinc can increase and aggravate the extent of brain damage if an ischemic or hypoxic crisis occurs, are directly applicable and highly troubling, when applied to elderly patients who are being urged to take pills with heavy dosages of zinc to help them protect their eyesight.

Any elderly person who is considering taking large dosages of zinc, and any physician who is deciding whether or not to recommend to elderly patients that they should take more than 70 mg of zinc per day, should be aware of the following comments by neurology researchers:

Choi et al, “Zinc and brain injury,” Annu Rev Neurosci 21: 347-75 (1998): “Growing evidence suggests that zinc may also be a key mediator and modulator of the neuronal death associated with transient global ischemia and sustained seizures, as well as perhaps other neurological disease states”.

Lobner et al, “Zinc-induced neuronal death in cortical neurons,” Cell Mol Biol 46: 797-806 (2000): “Although Zn2+ is normally stored and released in the brain, excessive exposure to extracellular Zn2+ can be neurotoxic. The purpose of the present study was to determine the type of neuronal cell death, necrosis versus apoptosis, induced by Zn2+ exposure. . . . These results suggest that Zn2+ can induce cell death with characteristics of either apoptosis or necrosis, depending on the intensity of the Zn2+ exposure.”

Sheline et al, “Zinc-induced cortical neuronal death: contribution of energy failure attributable to loss of NAD(+) and inhibition of glycolysis,” J Neurosci 20: 3139-46 (2000): “Excessive zinc influx may contribute to neuronal death after certain insults, including transient global ischemia. In light of evidence that levels of intracellular free Zn(2+) associated with neurotoxicity may be sufficient to inhibit [GAPDH, an enzyme that is crucial for energy metabolism] . . . Zn(2+) exposure also induced an early decrease in [NAD(+), an energy supply compound] levels, an event itself capable of inhibiting GAPDH.”

Lee et al, “Zinc translocation accelerates infarction after mild transient focal ischemia,” Neuroscience. 115: 871-8 (2002): “These data suggest that toxic Zn(2+) translocation, from presynaptic terminals to post-synaptic cell bodies, may accelerate the development of cerebral infarction following mild transient focal ischemia” (the term “infarction” refers to dead tissue; “cerebral infarction” refers to dead brain tissue, and irreversible brain damage).

Canzoniero et al, “Membrane-permeant chelators can attenuate zinc-induced cortical neuronal death,” Neuropharmacology 45: 420-8 (2003): “These data point to a potential therapeutic role for membrane-permeant Zn(2+) chelators . . . in attenuating neuronal death after certain acute insults.” (as mentioned above, zinc “chelators” bind tightly to zinc, in ways that can prevent the zinc from reacting with other molecules).

Ryu et al, “Depletion of intracellular glutathione mediates zinc-induced cell death in rat primary astrocytes,” Exp Brain Res. 143: 257-63 (2002): “In summary, reactive oxygen species generation, glutathione depletion, and mitochondrial dysfunction may be key factors in zinc-induced glial toxicity.”

Manzerra et al, “Zinc induces . . . up-regulation of NMDA receptor activity and excitotoxicity,” Proc Natl Acad Sci USA 98: 11055-61 (2001): “Functionally, this zinc exposure produced . . . an increase in NMDA receptor-mediated cell death. These observations suggest that the effect of synaptically released zinc on neuronal NMDA receptors may be biphasic: acute block, followed by Src family kinase-mediated up-regulation of NMDA receptor activity and cytotoxicity.”

Chen et al, “Zinc toxicity on neonatal cortical neurons: involvement of glutathione chelation,” J Neurochem 85: 443-53 (2003): “Several mechanisms have been implicated in pathological neuronal death including zinc neurotoxicity . . . Zinc caused neuronal cell death in a concentration-dependent manner . . . The results suggest that zinc non-enzymatically depleted GSH [the reduced form of glutathione, a tripeptide essential for proper mitochondrial functioning], an intrinsic factor for neuron survival, leading to activation of the cellular death signal and eventually neuronal death.”

Koh, “Zinc and disease of the brain,” Mol Neurobiol 24: 99-106 (2001): “Intracellular mechanisms of zinc neurotoxicity may include disturbances in energy metabolism, increases in oxidative stress, and activation of apoptosis cascades. Zinc inhibits [GAPDH enzymes] and depletes [NAD(+) and ATP, which are energy supplies]. On the other hand, zinc activates protein kinase C and extracellular signal-regulated kinase (Erk-1/2), and induces NADPH oxidase; these events result in oxidative neuronal injury. Zinc can also trigger caspase activation and apoptosis via the p75(NTR) pathway. . . . In addition to the neurotoxic effect, zinc may contribute to the pathogenesis of chronic neurodegenerative disease.”

These results and reports, taken together, offer strong and well-founded scientific evidence showing that high concentrations of zinc, in mammalian brains, can aggravate the amount of permanent brain damage that will be suffered, not by everyone, but by people who suffer from certain types of medical crises, such as strokes, cardiac arrest, or head injury.

Sadly, those types of crises are frequent among the same elderly populations that are most concerned about (and most vulnerable to) macular degeneration. Therefore, any product with heavy dosages of zinc that directly and openly targets elderly consumers raises serious and even grave questions about whether that product poses greater benefits, or greater risks, for such consumers.

Those questions and concerns become even more serious and pressing, when it is recognized that the increased risks of Alzheimer's disease or crisis-related neurotoxicity were not considered, recognized, or addressed by either (i) the vision experts who served on the “Age-Related Eye Disease Study Research Group”, or (ii) the researchers who coinvented U.S. Pat. No. 6,660,297 (Bartels et al 2003). To the best of the Applicants' knowledge and belief, these neurological concerns were never mentioned, discussed, analyzed, addressed, or given any consideration, of any sort, in any of the first eleven published AREDS reports, or anywhere in the patent application or file wrapper documents that led to issuance of U.S. Pat. No. 6,660,297 (Bartels et al 2003).

Potential concerns over zinc neurotoxicity were addressed, to a limited extent, in AREDS Report #12 (Yaffe et al 2004), which claimed that its results were “reassuring” that zinc was not having any adverse impacts on mental functioning. However, an analysis of that report reveals several major gaps and unanswered questions. For example, the data were based solely on people who completed a battery of mental tests, at the end of their participation in the AREDS trial; however, it did not say whether any mental testing was done near the start of the trial. A different abstract (Chew et al 2004, published separately but with overlapping authorship) disclosed that cognitive testing over at least a 2-year span was, in fact, being done on many participants; however, the authors of Yaffe et al 2004 chose to not disclose that such testing was being done, and they did not disclose any data from those tests. When they were asked by one of the inventors herein to disclose to him, in confidence, any preliminary data that might be available, or to have a simple computer routine written and run that would indicate whether any problems were evident, they refused both of those requests and said the data would not be available for at least another year.

Although Yaffe et al cited the Bush et al 1994 article on zinc and Alzheimer's disease, they did not cite or consider any of the articles that had shed more light on the Alzheimer's linkage during the decade since 1994.

It should also be noted out that 40% of the people who completed the AREDS trial did not complete the mental tests, and were excluded from the analysis that led to the reassuring comments by Yaffe et al. That raises an important question. If high zinc concentrations may trigger or accelerate Alzheimer's disease or other neurotoxic problems, as suggested by the reports cited above, then people who may have been adversely affected likely would not be able to complete the entire battery of mental tests that were given at the end of the trial; and, if that subset of people could not complete those tests, they were simply excluded from the data-gathering, and from the analysis. Clearly, if people who may have been adversely affected by heavy zinc intake were simply pushed off to one side and excluded from any analysis of the remaining data, that raises questions, since it would strongly bias any interpretations and conclusions toward a positive outcome, when the actual facts might indicate serious problems. However, that factor was not mentioned or addressed in Yaffe et al 2004.

Just as importantly, Yaffe et al did not cite or address any articles describing apparent links between zinc, and aggravated brain damage after a stroke or other crisis. Indeed, a longer unpublished version of the Yaffe et al manuscript actually cited Choi et al 1998, entitled, “Zinc and brain injury”, which contains statements such as, “Growing evidence suggests that zinc may also be a key mediator and modulator of the neuronal death associated with transient global ischemia and sustained seizures, as well as perhaps other neurological disease states”. However, Yaffe et al represented and described that article, not as an article filled with warnings about potential zinc neurotoxicity, but as being reassuring about zinc in the brain, since it indicates that zinc is already present in brain tissue.

Yaffe et al also it did not cite or address any articles that raise concerns about zinc and brain functioning, based on animal tests. As one example, Linkous et al 2004 reported that in laboratory animals, dietary intake of excess zinc led to elevated zinc levels in brain tissue, and to impaired mental performance in those animals. The authors of Linkous et al 2004, who were located by the inventors herein without difficulty because they are active in the field of zinc testing using animal models, were never contacted by the authors of Yaffe et al 2004.

In summary, AREDS Report #12 raised more questions than it answered.

AREDS Report 13 (published in May 2004, about six months before Report 12) was entitled, “Associations of mortality with ocular disorders and an intervention of high-dose antioxidants and zinc in the Age-Related Eye Disease Study” (Arch Ophthalmol 2004; 122: 716-726). It reported an apparently strong link in elderly people between antioxidant and zinc intake, and greater longevity. That report is complicated and requires careful analysis, but it generally indicated that elderly people with cataracts and macular degeneration tend to suffer from higher mortality rates, compared to people without such problems, presumably due to factors such as systemic damage due to oxidative radicals and inadequate vitamin and antioxidant intake. It also indicated that ingestion of the AREDS formulation tended to reduce rates of death that were not clearly attributable to known causes, such as cancer. Because of the potentially major implications of those findings, it is curious that they received almost no attention from the press. It must also be recognized that, if extra-heavy zinc ingestion can indeed trigger or aggravate Alzheimer's disease in some elderly consumers, an extended lifespan that only adds months or years of dementia to a slow form of death would not be much of a blessing or benefit, either to the patient, or to the patient's family and caregivers.

Other Potential Problems from Heavy Zinc Ingestion

In addition to the neurological risks cited above, still more reports indicate other concerns, risks, and issues raised by extra-heavy intake of zinc.

One set of concerns is raised by reports that when lab animals were fed heavy dosages of zinc over sustained periods of time, they developed cognitive impairments. Those animal tests are described in Linkous et al 2004, and Flinn et al 2005. The title of the Flinn et al paper is, “Enhanced zinc consumption causes memory deficits and increased brain levels of zinc,” and the abstract concludes with the statement, “These data show that long-term dietary administration of zinc can lead to impairments in cognitive function.” Additional unpublished data indicating similar results gathered by other research teams have been disclosed to the inventors herein, via personal communications, by researchers such as Dr. Ashley Bush.

In addition, Peto et al 2005 reported that a type of unwanted retinal debris called “drusen” contains unusually high levels of zinc, analogous to the high levels of zinc in beta-amyloid plaques in people with Alzheimer's disease. That report raises questions, including the question of whether one or more types of “zinc dyshomeostasis” may occur in some but not all elderly people, in ways that may trigger or accelerate the formation or growth of those deposits in some but not all elderly people who take large quantities of zinc.

As a third unrelated example, Leitzmann et al 2003 describes the largest and most powerful study ever done of a possible link between heavy zinc intake, and prostate cancer. In a survey of nearly 50,000 male health-care professionals over 14 years, men who ingested large dosages of zinc suffered from increased rates of prostate cancer.

To date, none of those reports or factors (several of which have come to light only recently) have been cited or addressed by the organizers of the AREDS trial, by the companies that sell AREDS products with heavy zinc dosages, or by the experts on the Food and Nutrition Board, at the Institute of Medicine. Clearly, the positions and opinions involving zinc intake that have been adopted by those people, companies, and agencies should be revisited and reconsidered, in view of these recent reports.

Institute of Medicine: RDA and “Tolerable Upper Intake” Levels

The official dietary and nutritional recommendations for zinc ingestion, which have been developed and published by committees of experts working under government supervision, deserve careful attention in any analysis of this subject. The starting point for analysis is to realize that the AREDS products being sold by B&L and Alcon to elderly consumers clearly exceed not just the “Recommended Daily Allowance” (RDA) levels, but also the “Tolerable Upper Intake” levels that have been established by impartial reviewers who are experts in nutrition and nutritional safety.

The RDA and “Tolerable Upper Intake” levels are determined and published by the Food and Nutrition Board, within the Institute of Medicine (IOM). The IOM is one of the branches of the National Academy of Sciences (NAS). Unlike the National Institutes of Health (NIH), which has thousands of employees and extensive laboratories, the IOM has only a relatively small staff. It works mainly through committees and boards of outside experts, most of whom work at universities or medical schools across the country.

The complete set of “Dietary Reference Intake” numbers for all vitamins, minerals, and other nutrients, can be downloaded from the IOM website, www.iom.edu, and from websites such as www.nal.usda.gov/fnic/, run by the Food and Nutrition Information Center in the Department of Agriculture. The most well-known category of “Dietary Reference Intake” (DRI) numbers are the “Recommended Daily Allowance” (RDA) numbers. Everyone has heard of these, since they appear on nearly all prepackaged foods. As mentioned above, current RDA values for zinc are 11 mg/day for adult males (age 19 and above), and 8 milligrams per day for adult females who are not pregnant or breast-feeding. Those numbers were revised in 2001; prior to that, 15 mg was the RDA level for both men and women.

As mentioned above, the IOM has also issued “Tolerable Upper Intake” levels (also referred to as “Upper Limit” values, abbreviated as UL) for most nutrients, including zinc.

Explanations of how the numbers were determined are provided in a book entitled Dietary Reference Intakes for Vitamin A, Vitamin K, Arsenic, Boron, Chromium, Copper, Iodine, Iron, Manganese, Molybdenum, Nickel, Silicon, Vanadium, and Zinc (2002 edition), published by National Academies Press. The chapter on zinc can be downloaded inexpensively, as a “pdf” file that can be read and printed using the Adobe Acrobat Reader software, from http://books.nap.edu/books/0309072794/html/442. html#pagetop.

The rationale for setting the “Tolerable Upper Intake Level” at 40 mg/day for zinc was explained on pages 481-488 of the chapter on zinc. Briefly, the Food and Nutrition Board focused on an enzyme called “erythrocyte copper-zinc superoxide dismutase”, abbreviated as ESOD. In in vitro assays, activity levels of this enzyme can provide an indicator of available copper concentrations, in circulating blood. Zinc and copper have long been known to share the same “uptake transport” system, which carries both minerals through the intestinal walls and into circulating blood. Since they share the same transport system, high dosages of zinc will compete against copper intake, and can inhibit the uptake of copper into circulating blood. Indeed, administering large dosages of zinc is the standard treatment for a rare syndrome called Wilson's disease, in which affected people cannot properly metabolize copper. By administering large dosages of zinc to patients who suffer from Wilson's disease, the uptake of copper (and the symptoms of excess copper accumulation) in people with that disease can be minimized.

The Food and Nutrition Board was aware that copper and zinc compete against each other for digestive uptake into circulating blood, so it evaluated the ESOD enzyme as an indicator to help it evaluate daily zinc dosages that would trigger decreases in ESOD activity (which, in turn, indicated that copper uptake was being hindered by high dosages of zinc). These tests indicated that 60 mg/day dosages of zinc were the level at which average people began to display some level of impairment in copper uptake. The Food and Nutrition Board then divided that 60 mg/day dosage level, which gave warning indications in average patients, by an “uncertainty factor” of 1.5. This factor provided an additional margin of safety, to ensure that nearly everyone (calculated to be at least about 98% of the public) will be safe and protected, regardless of normal genetic variability. Accordingly, when the 60 mg/day level (which triggered detectable adverse indications in average people) was divided by the uncertainty factor of 1.5, the resulting “Tolerable Upper Intake Level” for zinc was calculated to be 40 mg/day.

It should be noted that Bausch & Lomb and the National Eye Institute took the zinc-copper uptake competition into account, and added copper to the AREDS formulation that was tested (indeed, even the OCUVITE product being sold by Storz Ophthalmics in 1993 contained copper, to balance out the known competitive interaction between zinc and copper). However, Bausch & Lomb and the National Eye Institute failed to take into account a different factor: surplus copper poses a risk of aggravating the severity of brain damage, in people who suffer from Alzheimer's disease, since copper catalyzes the formation of peroxide compounds and oxidative radicals, within the sticky matrix that forms beta-amyloid plaques. Therefore, the addition of copper, to formulations containing heavy zinc dosages being taken by elderly consumers, may solve one problem, but it may also create or aggravate a different problem in people who have incipient, early, or mid-stage Alzheimer's disease.

A review of the chapter on zinc indicates that the experts on the Food and Nutrition Board of the IOM did not address (and therefore presumably were not aware of) any of the following factors: (1) the results of the AREDS-1 trial, which showed benefits of high-dosage zinc in preventing and treating macular degeneration; (2) the published reports cited above, indicating that zinc is involved in the formation and growth of β-amyloid plaques, in the brains of people with Alzheimer's disease; or (3) the published reports cited above, indicating that zinc may increase and aggravate brain damage after a stroke, cardiac arrest, or other crisis.

Since the nutrition experts at the IOM did not know of any of those factors, when they issued new dietary numbers for zinc in 2001, an important need has arisen for renewed attention to the question of optimal zinc dosages, especially among elderly people. Clearly, no elderly person wants to get macular degeneration, and slowly go blind; but just as clearly, no elderly person wants to get Alzheimer's disease, and become demented and unable to function.

Carotenoids in the Retina: Beta-Carotene, Lutein, and Zeaxanthin

Carotenoids are another important class of compounds that need to be considered, in any effort to develop nutritional supplements that can prevent or treat macular degeneration. Carotenoids were given that name because they were first isolated from carrots. They evolved in plants and in some types of bacteria, over millions of years, because they can absorb ultraviolet (UV) radiation without being damaged. Since UV radiation is toxic and even lethal to cells that are exposed to prolonged sunlight, carotenoids became crucially important to the survival of plants and microbes that gradually moved from submerged marine environments, to exposed land environments. Animals cannot synthesize carotenoids in their bodies. Therefore, they must ingest carotenoids as part of their diets. In this regard, carotenoids are directly comparable to vitamins, and can be considered as a class of vitamins.

The structures of several carotenoids that are relevant to eye care formulations are shown in FIG. 1 (which is prior art, since these molecular structures have been known for years). Several specific factors in these molecular structures are worth noting, as follows:

1. In the straight chain portion (between the two “end rings”) of each carotenoid shown in FIG. 1, the double bonds alternate with single bonds. That pattern of alternating single and double bonds is called “conjugation”. It is important, because when a series of single and double bonds are conjugated, the electrons that form bonds between adjacent atoms do not remain attached to specific atoms. Instead, the electrons become mobile, and they form an “electron cloud” that covers and surrounds the molecule. This same type of electron cloud also surrounds and stabilizes benzene rings and other aromatic molecules.

2. The conjugated electron cloud that surrounds parts of a carotenoid molecule is important, because it leads to remarkable results. First, when a carotenoid molecule is hit by ultraviolet light, the carotenoid will not break. Instead, the electron cloud is able to flex and yield, in a way that cushions and absorbs the blow. This is comparable to someone hitting wooden board and a rubber tire, with a sledgehammer. The board will break, because it cannot bend or deflect. However, a rubber tire will not break, because it can flex and yield in a way that allows it to absorb the force of the blow. When a UV photon hits a carotenoid molecule, the destructive power of that photon is used up and absorbed by the conjugated electron cloud. This prevents the UV photon from attacking and damaging other crucial molecules, such as strands of protein or DNA. By absorbing UV radiation, carotenoids protect DNA, proteins, lipids that form cell membranes, and other crucially important molecules in cells.

3. In addition to protecting molecules and cells against UV radiation, carotenoids are also anti-oxidants that can neutralize and quench unstable and destructive radicals. As discussed above, radicals are unstable because they have unpaired electrons. The presence of an entire movable cloud of electrons, surrounding a large carotenoid atom, gives it the ability either to receive and accept an unpaired electron from a radical that has an extra electron, or to donate an unpaired electron to a radical that needs one more electron. However, as mentioned above in the discussion of increased risks of lung cancer in smokers, under some conditions, some carotenoids (including beta-carotene) can reverse their anti-oxidant activity, and become pro-oxidants (i.e., they can accelerate the formation of destructive oxygen radicals).

4. Referring again to the structures in FIG. 1, zeaxanthin and lutein have “hydroxy” (HO—) groups attached to their end rings, while beta-carotene has no oxygen atoms or hydroxy groups. This leads to crucial differences in the way they are deposited and positioned in animal cell membranes, after they are eaten by animals. Since beta-carotene is made entirely of hydrocarbons, with no hydroxy groups, it is non-polar, which makes it soluble in oily liquids made only of hydrocarbons. Therefore, it will position itself in an animal cell membrane in the manner shown in FIG. 2, which depicts a beta-carotene molecule aligned in a manner that is roughly parallel to the surface of a cell membrane, sitting entirely within the center of the membrane. This results from the fact that animal cell membranes are made up of “bilayers” formed by molecules called phospho-lipids. These molecules will spontaneously line up together, when placed in a watery fluid, in a way that gives them a bilayer arrangement as shown in FIG. 2. Each phospho-lipid molecule has a polar (and therefore hydrophilic and water-soluble) “head” that contains phosphorous, bonded to a lipid (and therefore hydrophobic) “tail” made of hydrocarbons. Therefore, when phospho-lipid molecules form membranes, they create an oily hydrophobic layer in the middle of the membrane, flanked by water-soluble phosphate groups on both surfaces of the membrane.

As a result, as shown in FIG. 2, beta-carotene (an oily hydrocarbon with no oxygen atoms or hydroxy groups) will be deposited in the oily center of a cell membrane. By contrast, zeaxanthin and lutein (with hydroxy groups on both end rings) will be deposited in a manner that causes them to “span” or “straddle” the membrane, with their end rings protruding slightly beyond the inner and outer surfaces of the membrane. Because plants and animals evolved cooperatively, it is not just a coincidence that zeaxanthin and lutein have exactly the right length to span the thickness of animal cell membranes, with portions of their end rings sticking out from both sides of the membrane.

The most common fate of beta-carotene is that it is split in half, by enzymes, to release two molecules of a compound called retinol, also known as Vitamin A. The cleavage process involves hydrolysis, and it adds a hydroxy group to the end of the straight-chain portion of the compound that becomes retinol (Vitamin A). Retinol is converted by other enzymes into a similar compound called retinal, and certain straight-chain and bent-chain isomers of retinal are essential in the chemical reactions that enable rod and cone receptors (which are components of retinal neurons) to convert incoming light into nerve signals, which emerge from the retina and travel to the brain. Therefore, beta-carotene and Vitamin A are essential to vision, since they supply the precursors to the isomers of retinal that are crucial to rod and cone receptors, and nerve impulses.

Accordingly, beta-carotene plays an essential role in vision. Since it is also a known anti-oxidant, it was a good candidate for testing, to see whether high doses of beta-carotene could help prevent or treat macular degeneration. Therefore, high-dosage beta-carotene was included in the AREDS trial.

However, the results of the AREDS trial made it clear that beta-carotene, even when combined with Vitamin C and Vitamin E, did not provide any significant benefits against macular degeneration. By the late 1990's, it became clear for a number of reasons that high dosages of beta-carotene or Vitamin A would offer little or no serious hope for providing any significant benefits against macular degeneration, or any other serious eye disorders, among people who receive adequate baseline levels of vitamin A. Therefore, in the patent application that eventually issued as U.S. Pat. No. 6,660,297 (Bartels et al 2003), the inventors specifically stated that beta-carotene could be omitted, and replaced by lutein and/or zeaxanthin.

Lutein and zeaxanthin are of interest in efforts to prevent macular degeneration, because it has been known since the publication of Bone et al 1985 that those are the two carotenoids that give the macula its distinctive yellowish color. Several dietary surveys (notably including Seddon et al 1994) have indicated that diets rich in dark green vegetables (which contain lutein and zeaxanthin) appear to reduce the risks of macular degeneration.

However, after the Seddon report was published in 1994 and provoked increased interest in the field, it triggered a followup analysis, which involved blood samples that had been previously gathered from thousands of elderly people in the so-called “Beaver Dam Study” (named after Beaver Dam, Wis., a town that was chosen as the subject of a large multi-year study of eye health involving thousands of people). The results, published in Mares-Perlman et al 1995, were based on chemical testing of blood concentrations of lutein, zeaxanthin, and other carotenoids (such chemical tests are more reliable than memories and potentially biased, self-justifying answers to questions asked in a survey). The data and conclusions published in Mares-Perlman et al 1995 directly contradicted the conclusions of the Seddon 1994 report. In particular, that study clearly and directly stated, “Levels of the carotenoids that compose macular pigment (lutein with zeaxanthin) in the [blood] serum were unrelated to ARMD [age-related macular degeneration].”

In view of those conflicting reports and other problems, experts in eye research, and ophthalmologists who specialize in treating patients with serious eye problems, do not and cannot agree on the roles or potential benefits of lutein and/or zeaxanthin, in the retina. Evidence to support and prove this conclusion is available from numerous sources, both published and unpublished. Examples of such published reports by experts include Schalch 2000, and Jampol 2001.

Wolfgang Schalch is one of the foremost researchers in the world, in the field of carotenoids. For years, he has been one of the top carotenoid research managers at Roche Vitamins, the world's largest manufacturer and supplier of carotenoids (it should be noted that Roche Vitamins, a division of the Hoffman-LaRoche pharmaceutical company, was sold to DSM Chemicals in 2003). In a 2000 article, Schalch distinguished between epidemiological studies (which look for statistical correlations in populations based on past events, such as Seddon's dietary survey, and Mares-Perlman's blood sample analyses) versus intervention studies (which begin with populations that are divided into subgroups, which are then fed different dietary levels of compounds, and which are monitored for a period of time to see what effects the dietary differences have on the different subgroups).

Schalch 2000, at page 38, describes the serious problems that hinder and limit epidemiological studies, but then he also notes the difficulties that will arise if someone tries to carry out intervention studies using lutein and zeaxanthin. In Schalch's words, “Epidemiological studies therefore cannot provide definite proof of the efficacy of lutein and zeaxanthin in AMD. Such studies can provide evidence of possible relationships but cannot determine whether an effect is causal. The situation is different with intervention studies in which agents are administered on a double-masked, placebo-controlled, randomized basis and results are evaluated using predefined efficacy parameters. In the case of supplementation with lutein and zeaxanthin, where only small to moderate responses can be expected, only studies such as these [i.e., intervention studies] are likely to provide a definite answer as to an effect of lutein and zeaxanthin on AMD. However, the specific time-course and nature of this disease makes the design of such trials difficult.”

Lee Jampol is another world-class expert. He was chosen by the editors of the highly respected journal, Archives of Ophthalmology (published by the American Medical Association) to sort through the large amounts of data in the AREDS reports, and summarize and explain those findings to other physicians. Accordingly, Jampol wrote the commentary that accompanied and prefaced the two main AREDS reports (AREDS report number 8, on macular degeneration, and AREDS report number 9, on cataracts, both of which appeared in the October 2001 issue of Archives of Ophthalmology. In his 2001 analysis and commentary, Jampol stated on page 1534, “In view of previous studies suggesting that beta-carotene might be harmful in smokers and may be associated with a greater risk of lung cancer, beta-carotene should probably not be used by smokers and recent ex-smokers. An argument could be made that another carotenoid, lutein or zeaxanthin, could be substituted for beta-carotene, but the values and risks of other carotenoids [referring to lutein and zeaxanthin] is unknown at this time.”

Two years later, in November 2003, Jampol updated his analysis and commentary. In a commentary entitled, “AREDS—Two Years Later,” he stated, “Another important issue is the value of other carotenoids such as lutein. . . . Would it be more protective if another carotenoid (lutein) were used in place of the beta-carotene? . . . The answers to these questions await randomized trials. . . . The value of either of these carotenoids [lutein or zeaxanthin] remains unproven. . . . The value of lutein and zeaxanthin remains uncertain, although one or both of these carotenoids may be better than beta-carotene. Additional research is needed to answer this question . . . ”

Another example of how experts in this particular field of scientific and medical research regard lutein and zeaxanthin is provided by a major report compiled by a large panel of highly respected experts who specialize in retinal diseases. Those experts were brought together in 1998 by the National Eye Institute (NEI), and they were asked to develop strategic proposals and recommendations that would guide the NEI's funding for eye research over the next five years. The committee reviewed a wide range of options and candidate treatments, and in its report (contained as a entire chapter devoted solely to retinal diseases, in a published book entitled Vision Research) the committee specifically identified and named about 60 candidate treatments that the experts thought were deserving of careful scientific study and research funding. Even though that panel of experts identified nearly 60 specific research leads, it never even mentioned lutein or zeaxanthin.

It should also be noted that their omission of lutein and zeaxanthin from even a passing mention could not have been a mere oversight due to a lack of available information. Several of the experts who served on that committee had previously published articles that explicitly discussed lutein and zeaxanthin.

As another example of the uncertainties and doubts that surround lutein and zeaxanthin among experts who actually treat eye diseases, a patient suffering from wet macular degeneration, who was taking zeaxanthin capsules on a daily basis, was scheduled to have a photodynamic treatment using a drug called verteporfin (which is activated by a laser beam that is shone directly into the eye) at the Wilmer Eye Institute in Baltimore, which is affiliated with the Johns Hopkins School of Medicine. In late 2003, this patient told his ophthalmologist, one of the top experts in the world on treating wet macular degeneration, that he (the patient) was taking zeaxanthin. The ophthalmologist advised the patient to stop, since (in the opinion of the ophthalmologist) it probably would not help, and it might interfere with the verteporfin-laser treatment. Despite that suggestion, the patient continued taking zeaxanthin, up through the date of the treatment and continuing thereafter. The results of that treatment, which have been monitored for more than a year and a half as this is being written, have been outstanding, and have been much better than was expected by the ophthalmologist (that discovery is the subject of a separate patent application, and it has been supported by additional consistent evidence from other patients). For now, two points are worth noting. First, when one of the top eye experts in the world was advised that a patient suffering from wet macular degeneration was taking zeaxanthin, the expert advised the patient to stop taking it. Second, when the zeaxanthin discovery was disclosed in confidence to the two companies that sell verteporfin, both companies declined and refused to pursue, support, or fund even small-scale pilot tests to determine whether zeaxanthin will actually help improve the outcomes of photodynamic treatments using their drug.

Finally, it should also be noted that the National Eye Institute also has declined and refused to take any steps that would help advance or support trials of zeaxanthin. Despite specific and repeated requests from various entities, the NEI managers in charge of planning the second major AREDS trial (referred to herein as AREDS-2) have effectively refused to support any testing of zeaxanthin in any way that would enable it to be compared against lutein. Instead, since every commercially available supply of zeaxanthin (which is extracted from marigold flowers) contains a very small quantity of zeaxanthin (zeaxanthin content typically comprises only about 1 to about 4% of the lutein content), the NEI is making plans to test a lutein preparation containing a small quantity of zeaxanthin, and the design of the trial will prevent anyone from being able to identify and evaluate how much benefit (if any) was contributed by each of those two agents. That decision was confirmed in May 2005 by a high-level official of the NEI, at the annual conference of the Association for Research in Vision and Optics (ARVO).

Accordingly, an analyses of the roles of zeaxanthin and/or lutein in nutritional supplements for macular protection must take into account the facts described above, which can be briefly summarized as follows:
(i) recent reports by experts, published in respected and refereed journals, have directly stated that there is no solid and reliable evidence that zeaxanthin actually can or will help protect the retina;
(ii) published reports explicitly advise physicians who treat patients suffering from eye diseases that it is premature, and ill-advised, for any physician to instruct patients to begin taking any unproven and potentially dangerous supplements;
(iii) when a large panel of world-class retinal experts was asked, in 1998, by the National Eye Institute, to list the best and most promising candidate agents for future research to help prevent or treat retinal diseases, that entire panel, in its collective wisdom and expertise, completely omitted both zeaxanthin and lutein as candidates that should be considered for research, even though members of that panel were aware of zeaxanthin and lutein and some members had even published articles on them, prior to 1998;
(iv) in October 2003, when one of the world's top experts in treating macular degeneration was informed that one of his patients was taking zeaxanthin, the physician specifically advised the patient to stop taking zeaxanthin, since it might interfere with a different treatment that the physician was planning to give the patient; and,
(v) in 2005, the officials at the National Eye Institute who are in charge of planning and designing the AREDS-2 study decided to study both lutein and zeaxanthin, mixed together, in a formulation that includes five times as much lutein as zeaxanthin and that will block and prevent any researchers from being able to sort out and identify which agent is helping, and what their respective contributions are.

These factors offer powerful evidence that the invention disclosed herein, which regards zeaxanthin as an essential ingredient in multi-component formulations for preventing or treating eye diseases, is not obvious to those who are truly skilled in the art, and who in fact have devoted their careers to trying to prevent and treat eye diseases.

Patents on Nutritional Supplements

To complete an analysis of the art in this field, it must be recognized that hundreds of different dietary supplements, under thousands of brand and product names, are being marketed to the public in the U.S. and elsewhere, by means of advertising promises and claims suggesting that these products can help prevent or treat eye diseases, and maintain eye health. Faced with an overwhelming glut of competing promises and products, nearly all of which are unproven and many of which have only tenuous and flimsy support, it has become effectively impossible for people who are concerned about eye health to know which products will help, and which are merely preying on innocent victims whose vision is deteriorating, either because of general aging problems, or due to specific diseases, infections or injuries.

Those uncertainties and doubts extend to professionals who specialize in eye research or treating eye diseases. Even the most skilled optometrists and ophthalmologists simply do not know whether even the most well-known and widely-used nutritional supplements are actually safe and effective, in treating various eye diseases. This includes even the AREDS products; as cited above, a world-class reviewer recently stated, “The potential toxicity of both the AREDS formulation and these other preparations should be kept in mind. . . . Evidence supports the use of the AREDS supplements only in patients with intermediate or advanced AMD” (Jampol 2003). That position is frequently confirmed by comments from elderly patients and their families, and from skilled optometrists and ophthalmologists. They do not know, and cannot figure out from the huge surplus of information competing for notice and attention, which products (out of hundreds that are being sold and advertised) will truly help, and whether the potential benefits of any product are outweighed by the potential risks, either for all elderly people, or for elderly people who fall into certain categories or stages of eye diseases, or into fact patterns that do not fit cleanly into any simple categories. These problems are entirely consistent with the summary, provided above, of major and hugely important concerns over Alzheimer's risks and post-stroke neurotoxicity, which were never addressed (and apparently never even recognized) by the responsible officials at the National Eye Institutes, or by anyone working at the very large multi-national companies that sell the two main AREDS products. If one considers those problems at the highest levels of expertise and funding, and then expands those problems to include huge numbers of small companies trying to sell products over the unregulated frontier of the Internet, one can begin to realize that only a small fraction of the huge amount of information that is clamoring for notice and attention is actually safe, reliable, and useful.

Along those lines, just as many different nutritional supplement mixtures with various components have reached the marketplace by claiming to offer eye and vision benefits, many patents have been issued on combinations of potentially useful nutrients. It is impossible to create a complete and reliable list of patents in the field of eye-care nutrition, because most patents on nutritional supplements claim to provide health benefits in various areas other than eye care (such as for skin protection, cancer prevention, cardiovascular health, anti-aging, memory and mental functions, etc.), and because most such patents attempt to claim mixtures of nutrients regardless of their intended use (such patents may discuss eye benefits; they may allude to eye benefits in indirect but suggestive language; or, they may omit any mention of eye benefits, while referring to nutrients that are of interest herein). Nevertheless, the examples listed below can provide the reader with a sense of what has been claimed and asserted in this field.

U.S. Pat. No. 5,075,116 (LaHaye et al 1991) and U.S. Pat. No. 5,156,852 (LaHaye et al 1992), licensed to Alcon, claim methods for treating macular degeneration by a combination of: (i) a plurality of antioxidants, such as Vitamins C and E; (ii) a plurality of cofactors (such as zinc, copper, selenium, and manganese) that will activate metalloenzymes that will help scavenge and reduce oxidants; and, (iii) at least one “glutathione-elevating compound” such as L-cysteine, pyridoxine, and riboflavin.

U.S. Pat. No. 5,753,703 (Cavazza et al 1998), assigned to Sigma Tau, claims combinations of carnitine (a sulfur-containing amino acid that helps transport fatty acids into mitochondria, as discussed below) and an omega-3 fatty acid such as DHA (docosahexaenoic acid, also discussed below). Optional agents can be included, such as “vitamins, mineral salts, antioxidizing agents and vegetal fibers”. U.S. Pat. No. 6,365,622 (Cavazza et al 2002), also assigned to Sigma Tau, also claims combinations of carnitine and lipoic acid, but it is not clear how that patent is distinct from the Ames patent immediately below.

U.S. Pat. No. 5,916,912 (Ames et al 1999) claims “dietary compositions for enhancing metabolism and alleviating oxidative stress”, which contain carnitine and a “mitochondrially active antioxidant” that contains a thiol group, such as glutathione, N-acetyl cysteine, or lipoic acid.

U.S. Pat. No. 5,955,102 (Gorenbein et al 1999), assigned to Amway, claims softgel capsules containing DHA (the omega-3 fatty acid), lutein, and an “anthocyanoside” (this generally includes darkly-colored plant extracts, such as bilberry extract). This patent offers an example of a patent that completely fails to recognize the benefits of zeaxanthin over lutein; various claims in the patent indicate that lutein should be present in a concentration of “about 1 to 6 milligrams”, while zeaxanthin only needs to be present at a concentration of about 0.1 to 0.25 milligrams. Vitamins C, E, or A can also be included.

U.S. Pat. No. 6,417,233 (Sears et al 2002), assigned to Sigma Tau, claims mixtures of Co-Q10 (a coenzyme also known as ubiquinone, which helps support and stabilize mitochondrial functioning) and an omega-3 fatty acid (such as DHA). The mixture can also contain “vitamins, mineral salts, antioxidizing agents, amino acids, polysaccharides and vegetable fibers”.

U.S. Pat. No. 6,515,020 (Cavazza 2003), assigned to Sigma Tau, claims methods for treating memory and mental functions by administering combinations of carnitine and certain types of “stilbene” compounds (such as resveratrol) that can be extracted from various types of plants.

U.S. Pat. No. 6,572,899 (Gorsek 2003) offers an example of a patent that may or may not relate to retinal protection. It claims “healthy memory and neuro protection compositions”, and it is not clear whether “neuro protection” as used therein was meant to include retinal neurons. The composition claims mixtures of carnitine, ginkgo biloba, lipoic acid, blueberry extract, spinach extract, Co-Q10, phosphatidylserine, and phosphatidylcholine.

U.S. Pat. No. 6,733,797 (Summers 2004) also provides an example of a borderline patent that may or may not relate to eyes and vision. It claims, “A health supplement composition for mammals for improving memory and cognitive abilities”, and it is not clear whether vision should or should not be covered by phrases such as “cognitive abilities” or “cognitive improvement”, as used in that patent. Regardless, its claimed compositions include a phosphoester (such as phosphatidylcholine, phosphatidylserine, etc.) and a “herbal antioxidant” mixture that apparently must include each and all of barberry, bilberry proanthocyanidins, lemon bioflavonoids, lime bioflavonoids, orange bioflavonoids, curcuma, garlic bioflavonoid, ginkgo biloba, ginseng, gotu kola, grape seed proanthocyanidins, red apple quercetin, red onion quercetin, and Siberian ginseng. The mixture may also contain vitamins, minerals, and amino acids.

As stated at the beginning of this subsection, the list above contains examples of US patents on nutritional mixtures. This list is not exhaustive or comprehensive, and instead is intended to provide the reader with a sense of what has been published. As mentioned above, none of the components or mixtures listed above has had any major impacts in actually preventing or treating macular degeneration.

As the final item of background information, certain terms need to be defined and clarified. “Ocular” relates to the eye, and terms such as “ocular-active” can be used interchangeably with other terms such as ophthalmic, eye-related, vision-related, etc.

“Nutrients” as used herein refers to compounds that are found in a normal human diet and/or a healthy human body (including precursors and metabolites of nutrients that are actually in the diet), regardless of whether they are synthesized chemically, or extracted from natural sources. This term is intended to be distinct from drugs, pharmaceuticals, antibiotics, and other “xenobiotic” compounds that are not normally found in natural sources. “Nutritional supplements” as used herein is a subset of nutrients, and includes any naturally-occurring nutrients that have been either: (i) manufactured or formulated in a manner other than in a food source, such as in a tablet, capsule, syrup, powder, etc., or (ii) prepared and/or packaged as a food additive (either within a food product, such as in vitamin-fortified meat, dairy, or baked goods or beverages, or in “food-mixable” forms such as powders that are designed to be mixed with beverages). “Dietary supplement” is used interchangeably with “nutritional supplement” or “nutrient supplement”.

Accordingly, one object of this invention is to call attention to numerous published reports by numerous different research teams, indicating that serious risks of Alzheimer's disease, β-amyloid plaque formation, aggravated brain damage after a stroke or other crisis, and prostate cancer, are likely to be aggravated and worsened, if high dosages of zinc are specifically marketed to elderly consumers, in nutritional supplements advertised for treating or preventing macular degeneration.

Another object of this invention is to disclose a way to reconcile, balance, and find the best possible compromise between opposing and contradictory factors. On one hand, the AREDS-1 clinical trial proved that heavy-dosage zinc supplements, in conjunction with antioxidant vitamins, apparently can reduce the risk and the progression of macular degeneration. But on the other hand, heavy-dosage zinc supplements pose a major risk of contributing to the formation and growth of β-amyloid plaques, which accelerate the onset and worsening of Alzheimer's disease, not in all elderly people, but in some fraction of the elderly population that suffers from “zinc dyshomeostasis”. In addition, heavy-dosage zinc supplements apparently increase the risks of aggravated brain damage that may occur, not in all elderly people, but in those who suffer a stroke, cardiac arrest, or other medical crisis that directly affects the brain.

Another object is to provide eye care formulations that can be taken by elderly consumers with greater safety, efficacy, and benefits, compared to existing formulations containing heavy dosages of zinc that are potentially dangerous and neurotoxic to at least some elderly consumers.

Another object is to disclose a macular protection and eye health formulation that contains zinc, and that also contains one or more neuroprotective agents that can reduce the risks of zinc-related neurotoxicity and/or Alzheimer's disease, and which should also include zeaxanthin and/or β-cryptoxanthin, or be taken along with separate supplements containing zeaxanthin and/or β-cryptoxanthin.

Another object of this invention is to disclose ocular-active nutrient formulations that contain a combination of: (1) Vitamins C and E in relatively high dosages; (2) zinc, in fairly and reasonably but not dangerously high dosages; and, (3) at least one and preferably two or more mitochondrial stabilizing and/or neuroprotective agents, and which should also include zeaxanthin and/or β-cryptoxanthin, or be taken along with a separate supplement containing zeaxanthin and/or β-cryptoxanthin.

These and other objects of the invention will become more apparent through the following summary and description.

### SUMMARY OF THE INVENTION

Formulations that are marketed to elderly consumers for preventing or treating age-related eye diseases such as macular degeneration, and that contain substantial or high dosages of zinc, are modified in at least three and preferably four ways, compared to the formulations that were tested in the first “Age-Related Eye Disease Study” (AREDS-1) clinical trial. These modifications can reduce the risk of Alzheimer's disease and other neurotoxic damage in the brains of elderly people, who are the main consumers of products for preventing or treating macular degeneration.

Zinc has been shown to be heavily involved in the formation and growth of β-amyloid plaques, a key factor in triggering or aggravating Alzheimer's disease. Zinc also has been shown to increase the risk of aggravated brain damage during and after a crisis that deprives the brain of blood or oxygen, such as a stroke, cardiac arrest, or head injury. However, those factors were not recognized or considered by the people who organized and conducted the AREDS-1 trial, or by the companies that are now selling formulations with heavy dosages of zinc to elderly consumers, to prevent or treat macular degeneration. Accordingly, the neurologic risks created by extra-heavy zinc dosages (in products that arose from the AREDS-1 trial) are reduced, without losing the benefits of such treatments for preventing or treating macular degeneration, by a combination of at least two and preferably more modifications:
(1) the dosage of zinc is reduced to a maximum daily dosage of about 50 mg or less, preferably to a dosage of about 15 to about 40 mg/day; and,
(2) a substantial portion of any β-carotene in the formulation is eliminated, and replaced by zeaxanthin and/or lutein, at a daily dosage of at least 1 mg, preferably at least 3 mg, and even more preferably at least 5 mg, either in the same tablet or capsule, or in a separate supplement.

In addition, if desired, one or more compounds (such as Coenzyme Q10, carnitine, and/or a glutathione boosting agent such as N-acetyl cysteine) can be added that will boost and stabilize mitochondrial functioning, to help prevent and suppress apoptotic cell death.

The resulting formulations also should contain (or be administered in conjunction with) Vitamins C and E. Other active agents can also be included or coadministered if desired, such as: (1) lipoic acid, especially in its “R” (dextrorotatory) stereoisomer; (2) omega-3 fatty acids, such as docosa-hexaenoic acid (DHA); (3) taurine, also known as 2-amino-ethane-sulfonic acid; and, (4) β-cryptoxanthin, another carotenoid that has recently been discovered present at unusually high concentrations in the brain.

These improved combinations can provide safer yet more effective protection and maintenance of eye health and vision than other formulations currently available, especially among elderly patients who are at increased risk of Alzheimer's disease, strokes, cardiac arrest, and other medical problems.

In addition, now that the conflict and dilemma at the center of this invention (i.e., heavy doses of zinc may be good for aging eyes, but they are dangerous for aging brains) has been clearly identified and focused upon, it is hoped that this disclosure, which sits at the crossroads of two different problems that are both of great importance to elderly people, will lead to close and careful attention to this set of issues, by researchers, physicians, and nutritional experts who are affiliated with academia, medical schools, government agencies, not-for-profit foundations, and for-profit companies. In particular, officials and review panels at the National Institute of Medicine and the National Institutes of Health have been contacted and asked to revisit and reconsider all known factors and issues concerning zinc intake and supplements in the elderly, in view of recent discoveries concerning zinc and macular degeneration, zinc and β-amyloid plaques, zinc and ischemic or hypoxic neurotoxicity, zinc and prostate cancer, etc.

Finally, since aging dogs (which naturally generate β-amyloid plaques) offer a better animal model for studying Alzheimer's disease than mice or rats (since rodents do not naturally generate β-amyloid plaques), methods and approaches are proposed herein for establishing coordinated networks of “nursing homes” for severely aged dogs who have become incontinent, senile, or are otherwise approaching the natural ends of their lives. Such “canine nursing homes” can be staffed and operated by skilled supervisors and research managers, while much of the daily care and monitoring of prescribed diets and medicines can be handled by people who would enjoy the companionship of animals, and who may suffer from Down's syndrome or other disabilities that would not, however, prevent them from being good companions and caretakers for aging dogs. By coordinating and managing the testing of nutritional supplements and Alzheimer-preventive test drugs being fed to different groups of dogs, these networks of “canine nursing homes” can accelerate and improve the reliable and useful testing of nutritional supplements and Alzheimer-preventive test drugs, using animal models that are better-suited for such tests than rodents, while at the same time providing humane and benevolent alternatives to people who can no longer keep family pets that have become so old and infirm that euthanasia is being considered.

### BRIEF DESCRIPTION OF THE DRAWINGS

FIG. 1 (which is prior art) depicts the chemical structures of zeaxanthin and lutein (with an arrow pointing out the misplaced non-conjugated double bond in the “epsilon” end ring of lutein), and beta-carotene, a related carotenoid that does not contain any oxygen atoms or hydroxy groups.

FIG. 2 (which is prior art) comprises FIGS. 2A, 2B, and 2C. FIG. 2A depicts the bilayer structure of an animal cell membrane, formed by two rows of phospho-lipid molecules having water-soluble phosphate “heads”, and oily lipid “tails”. FIG. 2B depicts the way a molecule of beta-carotene (with no oxygen atoms or hydroxy groups) will aligned itself entirely within the oily interior of an animal cell membrane. FIG. 2C depicts how molecules of zeaxanthin and lutein will “straddle” or “span” an animal cell membrane, in a way that causes their end rings and hydroxy groups to protrude and extend out, beyond the cell membrane's outer and inner surfaces.

### DETAILED DESCRIPTION

As summarized above, this invention discloses improved formulations for preventing or treating age-related eye diseases such as macular degeneration, containing anti-oxidants, zinc, and at least one neuroprotectant. Because age-related eye problems occur mainly in people older than about 60, neuroprotectant(s) and zinc dosages in these formulations must be balanced in a way that will provide good benefits for eye health, while minimizing the neurologic risks that arise when elderly people ingest abnormally large dosages of zinc.

To understand this invention, one must recognize the problem and dilemma that are addressed by this invention. On one hand, in a major clinical trial that studied thousands of people (the AREDS-1 trial, designed and carried out by the National Eye Institute over a span of nearly 10 years), zinc at high dosages was clearly shown and proved to be beneficial for eye and vision health, among elderly consumers who are beginning to suffer from macular degeneration. That result was consistent with a number of smaller studies that previously had shown similar results.

However, the managers and researchers who organized and ran the AREDS-1 trial apparently were not aware of, and did not consider or address, two important neurologic risks and dangers that arise when elderly people take large and heavy dosages of zinc every day. Accordingly, those risks and dangers must now be recognized, confronted, and taken into account.

In particular, zinc has been shown to be directly involved in the formation and growth of β-amyloid plaques, a primary and even defining trait in Alzheimer's disease. In addition, cell culture tests and animal tests have shown convincingly that elevated zinc levels in the brain pose major risks of aggravating the extent and severity of permanent brain damage and impairment that may occur, if a person suffers a stroke, cardiac arrest, head injury, or other crisis that affects the brain.

Those two neurologic factors raise disturbing concerns and questions that cannot be ignored or dismissed, and the benefits of zinc, in protecting eyes and vision, must be balanced against the neurologic and other risks posed by daily ingestion of very heavy dosages of zinc, at levels much higher than the “Tolerated Upper Intake” levels recommended by nutrition experts.

Achieving that type of balance is the focus and goal of this invention, which has three major aspects: (i) recommending optimal zinc dosage ranges for widespread use by elderly consumers, which can provide useful and effective benefits for eyes and vision but which will not create excessively high Alzheimer, β-amyloid plaque, or other neurologic risks; (ii) providing a balanced formulation containing neuroprotectants that will further reduce the threat of zinc-related neurologic damage in elderly consumers; and, (iii) providing a balanced formulation that will provide substantially better protection against macular degeneration than was achieved by the antioxidants-plus-zinc combination that was tested in the AREDS-1 trial.

Zinc Dosages

As set forth above, one aspect of this invention relates to determining an optimal dosage of zinc, which takes fairly into account the Institute of Medicine's dietary recommendations, the results of the AREDS-1 trial, the correlation between zinc, β-amyloid plaques, and Alzheimer's disease, and published research findings on zinc and neurotoxic damage following a stroke or other crisis. Regrettably, the AREDS-1 trial tested only a single dosage of zinc, and did not study any additional dosages that could have helped generate a valid curve that would provide more insight into how different dosages of zinc would affect macular degeneration.

One of the challenges, in establishing optimal dosages of zinc that should be ingested by millions of people over the age of 60, is that those consumers do not all fit cleanly and neatly into a single, simple category. Instead, they will be distributed across statistical “bell curves” in a number of important respects (including age, body weight, overall medical condition, eye health, other medicines being taken, risk or history of Alzheimer's disease, and risk or history of stroke or cardiac arrest).

Nevertheless, a highly useful guiding principle can be safely relied on with respect to any and all such consumers. That principle is expressed and encapsulated in a phrase developed by economists, diminishing marginal utility.

The word “marginal” in this context is used in the same way as in “marginal tax rates”, which focuses not on the total taxes a person or company must pay, but on what will happen to the next dollar that is earned, if a person's or company's income increases. A “marginal” analysis does not focus on what happened in the past, or on numbers calculated as averages. Instead, it accepts whatever happened in the past as a fixed and unchangeable fact, and then it focuses on the question of what happens next, on a “going forward” basis. Instead of asking, “How much total good has this particular material provided to us?”, a marginal analysis asks questions such as, “Since we already have X amount of this material, how much more do we really need, and how much should we pay to get more? What will we do with the next batch that arrives?”

The reference to diminishing marginal utility reflects the fact that if a system is dynamic, adaptable, and somehow responsive and/or “intelligent”, then the first delivered quantity of some new and useful material can be put to very good use, to solve critical or even dire needs. However, after the most dire and pressing needs of a system have been met by making good use of a newly-delivered material, additional loads of the same material, delivered to the same location, organism, or system, will not provide as many benefits as earlier supplies.

Since this invention relates to nutrition, a simple example can be provided by food. If a man is hungry, the first plateful of food he gets will be greatly appreciated. The next plateful may still be good, but it won't be as good as the first. If he then eats a third plateful of food, it won't be because he's still hungry; and then, if he eats a fourth plateful of food, after already finishing three full plates, it's going to do him more harm than good.

As another simple example, every farm needs rain; it needs water, to enable crops to grow. However, too much rain leads to flooding, which can be every bit as destructive as drought.

The examples above illustrate both “diminishing”, and “marginal”, in the phrase, “diminishing marginal utility”. The marginal benefits that will be provided, by each additional load of material that is taken after one or more earlier loads have already been received, will decrease and diminish, in almost any imaginable system that is adaptive and responsive. In essence, diminishing marginal utility is a restatement of Aristotle's famous phrase, “Moderation in everything.”

There are several exceptions and qualifiers to the principle of “diminishing marginal utility”. However, in systems that are living, responsive, and adaptable, these exceptions usually operate only at the low end of the scale, and not at the upper end. Examples include systems that require a certain lower limit (often called a “threshold” or “critical mass”) to be met and then exceeded, before any substantial benefits will begin to accrue. As one example, when antibiotics are being used to treat a bacterial infection, no good will be accomplished at all, unless sufficient antibiotics are administered to reduce the bacterial population to a level where a person's immune system can take over and kill the rest. If low and inadequate dosages are used, they likely will do more damage than good, by creating conditions that select and promote the growth of antibiotic-resistant strains, which will then become even harder to kill.

However, that qualifier operates only at the low end of the curve, and the principle of “diminishing marginal utility” will take over again, once a “useful and effective” threshold is reached and passed. For example, if a week-long dosage of antibiotics, at a useful daily dosage, can help cure an infection, that does not mean a double-dosage will be twice as good for the patient, or that a triple-dosage will do three times as much good. Instead, super-heavy dosages typically do more harm than good, by causing digestive upsets, nausea, blurred visions, or other adverse side effects. The “marginal utility” provided by higher and higher dosages of antibiotics (or any other drugs) will decrease and diminish, until it drops quite literally into a negative zone, where additional quantities will cause more harm than good.

The reference above to “systems that are living, dynamic, and inherently responsive and adaptable” includes living organisms and ecosystems, while excluding, for example, structures that must be built in a certain and exact way. For example, if a construction company is building an office building that will be 30 stories tall, the builders will need a certain amount of steel, a certain amount of concrete, etc. On the lower and ascending side of the marginal utility curve, amounts that are less than needed will not be able to do “most of the good”; instead, they will simply leave the building unfinished. On the post-peak descending side of the curve, unneeded surplus amounts will not gradually decrease in value or utility; instead, after the necessary quantity has been reached, the value of any surplus drops almost immediately to zero, like a curve that has fallen off a cliff.

Accordingly, the principle of “diminishing marginal utility” must be understood and applied with due regard for its boundaries and limits. However, the boundaries and limits listed above do not apply to mammalian nutrition, since mammals are responsive and adaptive organisms that have evolved over billions of years in ways that enable them to sustain “homeostasis” (as described in the Background section, this refers to a responsive and adaptive equilibrium, which keeps operating parameters such as body temperature, blood flow rates, etc., reasonably constant and within functional limits, despite wide variations in surroundings and inputs). These types of homeostatic mechanisms show up fairly quickly, and are quite effective, if a person ingests too much of a certain compound that normally is a good nutrient, but which happens to be received in too great a quantity, on some particular day.

With regard to foods and nutrients, a marginal utility curve is initially steep, since foods and nutrients are necessary to avoid starvation, disease, and death. Then, once a substantial quantity has been ingested and the critical needs of the organism have been met, the marginal utility curve becomes less steep. At some point, the curve will flatten out, and it will reach a peak, hump, or plateau, where the maximal possible good has been achieved by that particular nutrient. After that point, the curve will turn negative, and any additional intake will mainly create a waste disposal problem, which the organism will need to take care of somehow.

That is indeed what happens with excessive zinc ingestion, and the process is controlled by various types of zinc-protein binding activities. In circulating blood, about 80 to 90% of zinc is either inside blood cells, or bound to proteins on the blood cell surfaces. In blood plasma, which contains about 1 to 1.3 μg/mL zinc, the large majority of soluble zinc is reversibly bound to various proteins, including albumin, alpha macroglobulin, and transferrin (e.g., Vallee 1988, Cousins 1989, and Vallee et al 1993). Those proteins provide a “buffer” or “temporary holding” capacity. If more zinc is needed by cells or other systems, proteins such as albumin, alpha macroglobulin, and transferrin can readily release the zinc they are carrying, and allow it to be taken by other proteins, including proteins that will transport the zinc into cell interiors.

However, if excess zinc is consumed (such as by people who take large quantities in vitamin supplements), a different process will be triggered. If zinc levels begin to increase beyond the normal homeostatic “set point” in circulating blood, various enzymes and genes will respond in a way that triggers the expression and production of “chelating” proteins, which bind much more tightly to free zinc. These “chelating” proteins notably include metallothionein (Sadhu et al 1990). These tight-binding proteins, secreted by organs such as the kidneys and pancreas in response to increased blood concentrations, help inactivate zinc in two different but complementary ways. Some of these proteins, secreted into the intestines, will bind to zinc while it remains in the intestines, thereby preventing it from crossing the intestinal walls and reaching circulating blood. Other proteins, secreted either into circulating blood or within kidney tissues, will grab zinc ions and transport them across the blood-filtering membranes in the kidneys, thereby causing the excess zinc to be secreted in urine.

The primary route of secretion of zinc is via feces, and fecal concentrations tend to be relatively stable, while urine concentrations can fluctuate more rapidly in response to elevated or reduced blood concentrations. Therefore, elimination in feces provides a fairly stable, constant, steady-state mechanism that handles the bulk of any surplus zinc, while elimination in urine provides a “fine tuning” mechanism that can respond more rapidly to brief fluctuations in blood levels.

Accordingly, the combined actions and principles of homeostasis and diminishing marginal utility offer a crucial and useful opening and opportunity, to help determine and provide optimal zinc dosages for elderly people who want, need, and deserve help in their efforts to prevent or treat macular degeneration, but who also do not want to get Alzheimer's disease or suffer from aggravated brain damage and permanent impairment if they have a stroke or other crisis.

The fact that a zinc dosage of roughly 80 mg/day was shown to be helpful in slowing the progression of macular degeneration (as shown by the AREDS-1 trial) does not mean or imply, in any way, that a dosage of only half as much zinc would provide only half as many benefits. Instead, because most of the oversupply generated by large zinc dosages will be eliminated in feces and urine, and because of how diminishing marginal utility actually works in living organisms, the much greater probability is that most of the benefits of zinc, in preventing or treating macular degeneration, can be provided by a supplement dosage in the range of about 15 to 30 mg/day. Increasing those dosages to higher but not dangerously high levels, it is likely that the large majority (roughly estimated to be in the range of about 70 to 90%, or possibly even 95%) of the benefits that might be provided by a 70 to 80 mg daily dosage, in preventing macular degeneration, could be provided by a dosage of only half that much, in a range of 35 to 40 mg/day (which, it must be kept in mind, is still quite a bit higher than most elderly people normally receive each day).

It should be kept in mind that 40 mg/day has been designated as the “Tolerable Upper Intake Level” for zinc, as determined by nutritional experts who extensively studied the biochemistry and physiological effects of zinc. However, it also should be noted that those experts did not address and apparently did not know about (i) the neurological risks of zinc in elderly people who suffer from or are at elevated risk of Alzheimer's disease, and (ii) the risks that excessive zinc may cause increased brain damage, in elderly people who suffer from strokes, cardiac arrest, or other crises. Accordingly, the likelihood is substantial that the “Tolerable Upper Intake Level” would have been set at a substantially lower level, if not for all adults, then at least for people over the age of about 60 or so, if the experts at the Institute of Medicine had known about the apparent connections between zinc, Alzheimer's disease, and excitotoxic brain damage after an ischemic or hypoxic crisis.

Decisions to reconsider (and possibly revise) the “Tolerable Upper Intake Level” for zinc can be made only by the members of the Food and Nutrition Board, at the Institute of Medicine. Accordingly, the Inventors herein have taken steps to ensure that all members of that Board, and any staff members at the Institute of Medicine who work with that panel of experts, are advised of the articles cited herein concerning: (i) the benefits of high-dosage zinc, in preventing or treating macular degeneration; (ii) the role of zinc in contributing to the growth of β-amyloid plaques, in Alzheimer's disease; and (iii) the risks of elevated zinc levels causing aggravated brain damage, in a person who suffers a stroke, cardiac arrest, or other crisis.

As with any government panel, their deliberations may take years. In the meantime, the Inventors have set forth herein a set of recommendations, for consideration and evaluation by panels of nutritional experts, and by physicians, ophthalmologists, and optometrists who must make decisions today and in the near future on what their patients should do while waiting for better guidance, and by elderly consumers, family members, and friends who want to help them and do what is best for them. Clearly, extensive and careful review by experts who are aware of all relevant factors is needed, and will ultimately provide the best long-term guidance. But just as clearly, physicians, relatives, and care-givers who work with elderly people need good and useful guidance on how to advise those patients, today, without waiting for years for some government agency or panel to slowly and exhaustively review a pressing problem. Somehow, people who understand the problems, and the factors involved, must be encouraged and even driven to come up with the best guidance they can offer, today, for elderly consumers who are already actively sliding down either or both of two slippery paths, one leading to macular degeneration and blindness, and one leading to Alzheimer's disease and dementia. People who are responsible for giving the best possible care and medical advice to elderly people simply cannot wait for years, to find out what they should begin doing tomorrow, next week, or next month.

In view of all known relevant factors, and unless and until clinical trials are carried out to specifically measure and establish dose-response curves in elderly patients who fall into various nutritional, age, body weight, or other categories, the Applicants herein have concluded that, in eye-care formulations that are marketed primarily to elderly consumers, the following guidelines should be followed.

(1) When taken on a preventive (prophylactic) basis, to sustain good eye and vision health, in the absence of a diagnosed disorder (or a loss of vision clarity that has become apparent to the person), daily dosages of zinc generally should not exceed about 30 mg/day (all dosages herein are expressed as elemental zinc, rather than as zinc oxide, gluconate, etc.). Daily dosages of 40 mg should be regarded as a maximum tolerable level, if supplemented by a copper dosage of about 0.5 to 1.5 mg.

(2) When taken on a therapeutic basis, by a person with a diagnosed eye or vision disorder, or a person who has suffered a noticeable decrease in vision clarity that cannot be corrected by lenses, a series of two different dosage regimens is recommended. The first stage can be regarded as a “rapid buildup” or “crisis response” stage. During that period, which generally will last from about 2 to about 8 weeks, a person with a known eye disorder will need to reach higher blood concentrations of zinc, as quickly as possible. Accordingly, during that stage, the recommended dosage will be in a range of about 30 to about 50 mg/day, depending on body weight (30 mg/day is recommended for an adult with a body weight ranging from about 50 to 60 kilograms, or about 110 to 130 pounds, while higher dosages are recommended for larger and heavier adults). After an initial “rapid buildup” regimen is completed, the person should then take somewhat lower long-term zinc dosages, designed to sustain blood concentrations at levels higher than were present when the eye or vision problem was first noticed. Preferred dosages should be determined by evaluating blood tests, for each specific patient; however, among people who cannot afford such tests (this class is the most likely to suffer from poor nutrition, and the most likely to impose the heaviest burdens on society if they become functionally blind or demented), preferred zinc dosages for long-term ingestion, to help prevent a known vision problem from worsening, generally will be in a range of about 20 to 40 mg/day, depending on age, body weight, and other factors.

In order to prevent the neurologic risks of these zinc dosages from outweighing their advantages in protecting eye and vision health, these dosages of zinc preferably also should be accompanied by at least one and preferably two or more types of neuroprotective agents, as discussed below.

It also should be emphasized that the dosage ranges recommended above, for zinc, should be evaluated in clinical trials that also administer zeaxanthin (and preferably β-cryptoxanthin as well) to all test subjects, at dosages of at least 3, preferably at least 5, and even more preferably at least 10 mg per day. For reasons described below, the Inventors herein believe and assert that:
(1) zeaxanthin very likely is the true key to preventing and treating macular degeneration;
(2) any other agents (including zinc) that are taken to prevent or treat macular degeneration will have only limited and indirect benefits, unless accompanied by zeaxanthin;
(3) the best way to determine truly optimal zinc dosages for preventing or treating macular degeneration is by testing zinc at 20 to 40 mg/day, combined with zeaxanthin at dosages of at least 5 and preferably 10 or 20 mg/day.

Reasons and factors supporting these assertions are provided below.

Comparisons Between Zeaxanthin and Lutein

Zeaxanthin and lutein are the two carotenoids that give the macula its yellow color; Because lutein can be extracted in bulk from the bright orange flowers of certain strains of marigolds, it has been commercially available for at least two decades, and it is widely used as a pigmenting additive, in animal feeds that are fed to chickens or farm-raised salmon.

However, because of certain molecular, anatomic, and medical factors, zeaxanthin is believed to be strongly preferable over lutein, for protecting eye and vision health, and for preventing and treating macular degeneration in particular. Indeed, zeaxanthin is regarded by the coinventors herein as being the most promising agent ever discovered, for preventing and treating macular degeneration.

The use of zeaxanthin for treating macular degeneration is described in some detail in U.S. reissue Pat. No. Re-38,009 (Garnett et al 2003, coinvented by the first named inventor herein). It can be directly included in tablets, capsules, fortified foods or drinks, or other formulations that contain additional ocular agents; or, it can be orally ingested separately, such as in 10 mg capsules sold by ZeaVision LLC (www.zeavision.com). As discussed below, any such zeaxanthin preferably should be in the form of the 3R,3′R stereoisomer, as found in natural dietary sources.

To understand the contributions of lutein and zeaxanthin in eye health, a number of factors must be considered. Although nearly all the facts discussed below can be gleaned from various articles (if one knows where to look), many of these facts are obscure, and are known mainly to specialists in plant biology or carotenoid chemistry, rather than ophthalmologists, optometrists, or vision researchers.

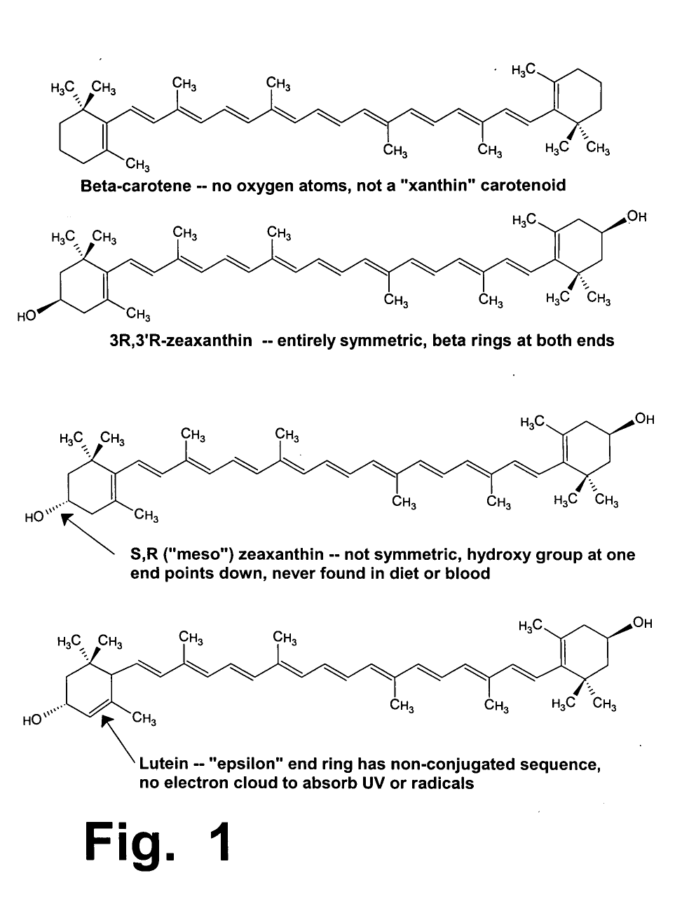

One major difference between zeaxanthin and lutein is that zeaxanthin has a higher degree of electron “conjugation” than lutein. When double and single bonds between adjacent carbon atoms are placed in an alternating sequence, as shown in the structures drawn FIG. 1, the electrons that form those bonds gain the ability to move and migrate, in a cloud-like structure, rather than being pinned down in a specific location between two carbon atoms. This movable and flexible electron cloud can absorb ultraviolet photons without being damaged or broken apart, in a manner analogous to a rubber tire absorbing a hammer blow without breaking.

The electron cloud also is what neutralizes and “quenches” oxygen radicals. Radicals are unstable and destructive because they have unpaired electrons. Since a carotenoid molecule having 40 carbon atoms is nearly surrounded by a large, flexible, adaptable cloud of mobile electrons, it can either donate an electron to a “singlet” radical that needs an electron, or it can receive a surplus electron from a “triplet” radical that has one too many electrons.

These factors explain the importance of zeaxanthin having more, longer, and better conjugation than lutein. The only difference between them is in the location of a certain double bond in one of the two end rings, as shown by the arrow in FIG. 1. Zeaxanthin has two identical end rings, both with “beta” structures, while lutein has only one “beta” end ring, while its other end ring has an “epsilon” structure. In zeaxanthin, the double bonds in both “beta” rings are positioned in a way that continues and extends the protective conjugated electron cloud over a portion of both of zeaxanthin's two end rings. By contrast, in lutein's “epsilon” ring, the double bond is misplaced, in a way that does not support or allow conjugation. Therefore, lutein has no UV-protective, radical-quenching electron cloud over one of its two end rings.

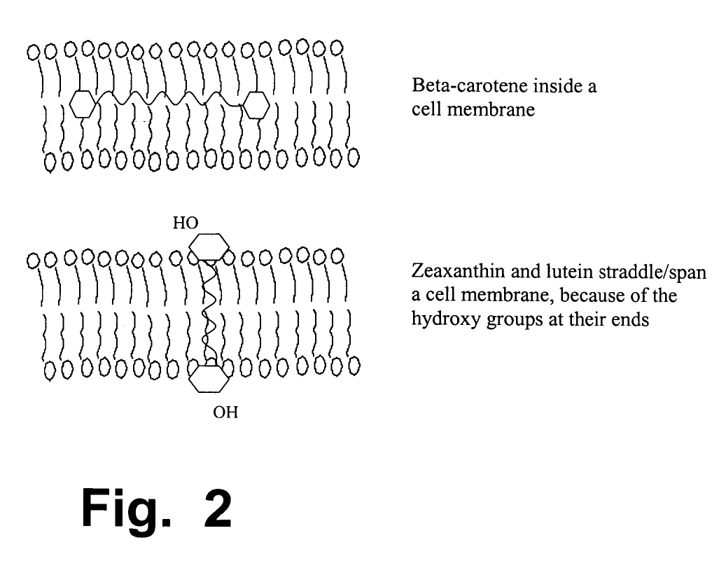

This defect in the non-covered and non-protective “epsilon” ring of lutein becomes even more important because of how lutein and zeaxanthin are deposited and positioned in an animal cell. As illustrated in FIG. 2, both molecules are positioned in a way that causes them to straddle the thickness (width) of an animal cell's outer membrane. This positioning results from how carotenoids interact with animal cell membranes. As described in any textbook on cells or physiology, animal cell membranes are formed from phospholipids, which are long molecules with a “head” that is hydrophilic (water-soluble), bonded to a “tail” that is oily and hydrophobic. When placed in water, these molecules will spontaneously line up in “bilayer” spheres, with the outer and inner surfaces covered by the water-soluble “heads”. The oily “tails” try to minimize their contact with water, so they line up in a way that forms an oily center layer inside the membrane.

Zeaxanthin and lutein both have oily and hydrophobic straight-chain portions, connecting their end rings. This straight-chain portion, in both molecules, will come to rest inside the oily center layer inside an animal cell membrane. However, the end rings of zeaxanthin and lutein have hydroxy groups attached to them. These hydroxy groups are hydrophilic, and they seek contact with watery fluids.

As a result, zeaxanthin and lutein will be positioned in a way that causes the tips of their end rings to extend and protrude outwardly, from both the interior and exterior surfaces of cell membranes. It is no mere coincidence that zeaxanthin and lutein have molecular lengths that are perfectly suited for that type of membrane “straddling” (or spanning) position, with only parts of their end rings sticking out and accessible. Plants and animals coevolved over the eons, and zeaxanthin and lutein were selected for that type of membrane-straddling positioning, in animal cells, because they have exactly the right lengths.

The “straddling” or “spanning” orientation of zeaxanthin and lutein, in animal cell membranes, explains why the minor structural difference between them becomes crucially important. Zeaxanthin's conjugated electron cloud covers part of both of its end rings; therefore, it can extend a UV-protective, radical-quenching electron cloud out beyond both surfaces of an animal cell membrane. By contrast, as noted above, lutein's “epsilon” end ring has a misplaced double bond, which disrupts and prevents the electron cloud from covering part of its epsilon end ring. Since the electron cloud is crucial for absorbing and protecting against UV radiation and destructive radicals, the tiny structural difference between lutein and zeaxanthin becomes crucially important in how they actually perform, after being eaten by animals.

Two other factors also should be noted, to distinguish zeaxanthin from lutein. First, zeaxanthin is perfectly symmetrical, end-to-end. Both of its ends are entirely identical, in all respects. It does not matter which end of a zeaxanthin molecule happens to be “grabbed” by an enzyme that will insert the zeaxanthin molecule into an animal cell membrane.

By contrast, lutein is not symmetrical. Its two ends are different from each other. It is not fully known how that lack of symmetry affects the placement of lutein in animal cell membranes, but it certainly cannot be helpful or beneficial. This likely is one of the factors that causes zeaxanthin to be deposited into the crucially-important center of the macula, while lutein is deposited at lower concentrations in the center, and at higher concentrations around the less-important outer portions of the macula.

The final factor that deserves mention and explanation is this. Lutein is much more abundant than zeaxanthin, in plants. Even in dark green vegetables with relatively high natural zeaxanthin content (such as spinach and kale), lutein is present at concentrations that range from about 20 to more than 100 times greater than zeaxanthin. Since one of lutein's end rings is exactly the same as zeaxanthin's end rings, that is a curious and unusual fact; clearly, any plant cell has the necessary equipment to make zeaxanthin, so why don't plant cells make more zeaxanthin?

The answer is fairly straightforward, but it is known only by botanists and a few other specialists. The positioning of the non-conjugated double bond in lutein's epsilon ring gives lutein a slightly “kinked” (or bent) configuration, at that end of the molecule. That kinked and bent structure allows lutein to fit into circular “light-harvesting” structures that are found in chloroplasts, which carry out photosynthesis in plants. Therefore, lutein is much better than zeaxanthin at helping plants carry out photosynthesis. This explains why plants evolved in ways that heavily favor the production of lutein over zeaxanthin. Actually, zeaxanthin also occurs in nearly all plants, but only in tiny quantities, and the molecule shuttles back and forth between zeaxanthin and “violaxanthin”, a similar carotenoid, as part of the day-night cycle of plant metabolism. Therefore, zeaxanthin cannot accumulate in significant quantities, in most plants, while lutein does, because of the essential role it plays in photosynthesis.

However, photosynthesis does not occur in animals, and lutein has no particular advantages after plant material has been ingested by animals. Rather than allowing it to fit properly into circular “light-harvesting” structures in chloroplasts, lutein's lack of end-to-end symmetry, and the kinked/bent attachment of the epsilon ring to the straight chain, become problems, rather than advantages, in animal metabolism. Those problems hinder lutein's ability to fit properly into animal cell membranes, and they hinder, reduce, and limit the protective benefits that lutein molecules can provide for animal cell membranes.

For all of these reasons, zeaxanthin is believed by the coinventors to be substantially better than lutein, in preventing or treating macular degeneration.

This conclusion and belief is also supported by a fact mentioned above. The molecular differences between zeaxanthin and lutein have led to an important and revealing difference between the way human retinas deposit zeaxanthin versus lutein. In human eyes, zeaxanthin is deposited at highest concentrations in the crucially-important center of the macula. By contrast, lutein is deposited at lower concentrations in the center of the macula, even though it is available in food sources at concentrations that are many times greater than zeaxanthin.

Indeed, the human retina even attempts to convert lutein into zeaxanthin. This conversion process apparently uses light-triggered and/or enzymatic processes that are not fully understood. However, that process cannot convert lutein into the normal form of zeaxanthin found in nature (the 3R,3′R stereoisomer); instead, it converts lutein into a different and highly unusual stereoisomer. One end ring has the conventional “R” configuration, but the second end ring has an unnatural “S” configuration that is not found in any dietary sources. The S-R isomer is often called meso-zeaxanthin, and it is discussed below.

Zeaxanthin also has been found to enter brain tissue, in unusually high quantities, in both animals and humans.

Japanese quail have become an accepted animal model for testing candidate retinal protection compounds, because their retinas deposit and contain both zeaxanthin and lutein. By contrast, mammals such as rodents, cats, and dogs do not deposit or metabolize carotenoids in the same ways humans do, and do not have maculas. Tests on Japanese quail (described in Thomson et al 2002 and PCT application WO 97/16175) indicated that when zeaxanthin was fed to the birds in a controlled diet, it permeated through their blood-brain barriers and entered BBB-protected brain tissue.

Similarly, Craft et al 2004 indicated that in elderly humans (based on autopsy analyses), zeaxanthin was deposited into brain tissue in substantially higher concentrations than would be expected, based on dietary intake and blood concentration.

Because of the known anti-oxidant properties of zeaxanthin, it is believed by the inventors that zeaxanthin acts as a highly useful neuroprotectant, in brain tissue. Accordingly, zeaxanthin is believed by the coinventors to be likely to reduce any neurotoxic risks that may be created by large dosages of zinc, in any macular protection products as described herein.

It also should be noted, as reported in Craft et al 2004, that beta-cryptoxanthin (a carotenoid found mainly in fruits, rather than green vegetables) was also present in surprisingly high concentrations, in human brain tissue.

Those data, and the patterns of concentrations of zeaxanthin and beta-cryptoxanthin that were found in different regions of the brain in elderly patients, support the conclusion that zeaxanthin and beta-cryptoxanthin are likely to be providing useful and protective benefits, in human brains. It should also be noted that even though lutein is at least 50 times more prevalant than zeaxanthin in typical diets, the brain somehow manages to sequester and deposit almost as much zeaxanthin as lutein, in all human brain regions that were tested. This is regarded as additional evidence that CNS tissue prefers zeaxanthin over lutein, and that zeaxanthin is likely to offer better benefits than lutein, for neurons and CNS tissue.

Based on the known functions and roles of carotenoids as anti-oxidants, the discovery and presence of unusually high levels of both zeaxanthin and beta-cryptoxanthin, in human brain tissue, leads to the belief and assertion by the inventors that: (i) zeaxanthin and beta-cryptoxanthin are performing useful and beneficial roles inside the brain, and (ii) out of all the known carotenoids, zeaxanthin and beta-cryptoxanthin offer the best promise of actual benefits when use in nutritional supplements, especially in nutritional supplements developed for elderly consumers. Dosages of at least 3 mg/day, preferably at least 5 mg/day, and even more preferably 10 or more mg/day (each) of both zeaxanthin and β-cryptoxanthin are preferred ingredients in formulations sold for human use (especially for elderly consumers, and especially in products that contain 20 mg/day or more of elemental zinc), regardless of whether such formulations are labeled, advertised, or marketed for macular protection purposes. While such products may include lutein if desired, it is believed that such products are likely to perform better if lutein is excluded entirely, or limited to no more than about 3 to 5 mg/day, to minimize problems of lutein competing against zeaxanthin and beta-cryptoxanthin for digestive transport and uptake into circulating blood.

Carotenoid Benefits Beyond Protection from UV and Radicals

In addition to the foregoing, the inventors herein also have noticed various correlations and previously unconnected data points that tend to suggest that zeaxanthin and beta-cryptoxanthin in particular (and other carotenoids in general) may also be able to help suppress and control various inflammatory steps and pathways.

One of the factors that led the first-named inventor herein to recognize that a carotenoid such as zeaxanthin is both (i) a crucial ingredient that is essential for eye health, and (ii) an “anchor” ingredient that can enable other useful agents to work more effectively and in a synergistic manner, was the gradual realization, which arose over a span of more than a decade of reading thousands of articles, abstracts, and patents on carotenoids, of how many different roles carotenoids can play. In particular, carotenoids can play any or all of several different roles that extend above and beyond their well-known roles in absorbing ultraviolet radiation and quenching oxygen radicals. Since most medical researchers and ophthalmologists apparently are not aware of the activities and factors listed below, or have not yet recognized how these numerous contributing activities and factors cumulatively enable zeaxanthin to provide a remarkable range of benefits to people suffering from eye problems, a numbered list is provided, directly below, which briefly touches on half a dozen of the lesser-known activities of carotenoids.

One of the factors that has caused these activities to be overlooked and ignored, by medical researchers, is that all of these activities can generally be described as offering only mild, weak, and partial levels of benefit. Therefore, when it comes to matters such as designing, organizing, and paying for clinical trials to prove that these benefits can be important, these potential contributing factors fall into a highly doubtful and unreliable zone, where they do not receive serious attention. This factor is aggravated by certain types of biases that are built into clinical trials, including factors that center around what is called the “standard of care” for any particular type of disease or disorder that is being tested. Briefly, the “standard of care” issue, which arises in designing and conducting clinical trials, generally means that it is unethical, and often even illegal in ways that can lead to lawsuits and huge damage awards, for a company to withhold from patients a treatment that is known to be effective in treating a certain type of disorder that such patients may be suffering from.

As an illustration of this doctrine, it would be effectively impossible to carry out a human clinical trial to prove that carotenoids have a relatively low yet beneficial level of activity in helping prevent or control ocular inflammation in humans, because there are other known treatments (mainly anti-inflammatory steroids) that are much more targeted and potent in treating that particular problem. To properly test carotenoids for anti-inflammatory effects in humans, the best known treatments would need to be withheld from all patients being tested, including “control” patients who would receive nothing but ineffective placebos to establish comparative data from untreated subjects. However, in tests on humans suffering from a serious problem that has a good and effective treatment, withholding known effective treatments even from “control” patients would be unethical, and improper. This effectively makes it impossible to carry out a clinical trial to prove that some carotenoid can provide a mild but potentially useful benefit against a serious problem (such as treating ocular inflammation, to continue the example offered above), when other agents are already known to be more effective in treating that problem. For those and other reasons, physicians, scientists, and other experts regard the factors listed below as being unproven and unreliable, without sufficient support to extrapolate any data from cell culture or animal tests to actual human medicine. Therefore, in the consensus view of most physicians, scientists, and other experts, the beliefs set forth below, no matter how sincere they may be, are not adequate to support medical recommendations by physicians who must diagnose and treat patients suffering from macular degeneration or other eye or vision problems.

The inventors herein are aware of the difficulties in proving that zeaxanthin can provide the types of useful but relatively mild and weak benefits described below. However, they have recognized how these types of relatively mild and weak benefits can be woven and incorporated into a larger package of active agents that, together, can provide significantly stronger benefits that will be able to overcome and surpass the hurdles and requirements that apply to human clinical trials.

In addition, it should be noted that various articles describing apparently unconnected aspects of carotenoids gradually accumulated, in the overall understanding and perspective of the inventors, until they led to an insight and recognition that has never been suggested or addressed in any prior art. Accordingly, the information below, on a number of relatively weak activities of carotenoids, is regarded as part of this invention, and it is suggested and taught herein that these factors, taken together, must be combined and connected into larger cohesive framework that merits serious and careful attention by physicians, ophthalmologists, and optometrists.

Accordingly, the following factors, all of which led to a specific insight that supports and substantially contributes to this invention, need to be recognized and given due consideration:

(1) Carotenoids have a mild ability to help control and reduce inflammation, as described in articles such as Ohgami et al 2003, Lee et al 2003, Gonzalez et al 2003, Ford et al 2003, and van Herpen-Broekmans et al 2004. Although their effects in this area are not as potent as anti-inflammatory steroids, these effects may nevertheless become important, in significant numbers of patients suffering from eye disorders, because any inflammation that involves or affects either or both eyes is an important threat and risk factor, and can lead to serious and even severe adverse results, including blindness.

Even a relatively slight episode of inflammation, if it directly affects either or both eyes, can permanently damage the eyesight, if the inflammation leads to increased fluid pressure involving the vitreous humor (i.e., the jelly-like clear liquid between the lens and the retina). Except in the small macular region at the center of the retina, the capillaries that serve the retina actually sit on the front surface of the retina, directly exposed to fluid pressures, rather than being embedded within a structural layer. Therefore, if fluid pressures increase in the vitreous humor, those elevated fluid pressures will press directly against the exterior surfaces of the retinal capillaries. Since capillary walls must be extremely thin, to promote rapid exchange of oxygen, nutrients, and metabolites, they cannot resist and push back, if elevated fluid pressures are imposed on them. Therefore, following basic principles of fluid flow, even a slight elevation in the fluid pressure of the vitreous humor, in an eye, can cause a significant portion of the blood that normally flows through retinal capillaries to be diverted. The blood supply that the retina needs will simply take a different route, somewhere else in the body, at any other location where the capillaries are not being squeezed and compressed.

This mechanism explains why glaucoma will cause blindness if not treated. Glaucoma is actually the name given to an entire class of eye diseases that share a common trait: they involve elevated fluid pressures inside the eye. This pressure elevation can be caused by any of several factors (such as the secretion of too much fluid by certain types of eye tissues, factors that hinder drainage and flow through the drainage ducts, etc.). Regardless of the cause, any disorder that involves chronic elevated pressure inside the eye is called glaucoma, and it will lead to blindness, since elevated pressures will hinder the flow of blood through the retinal capillaries.

If elevated fluid pressures inside the eye are caused by inflammation due to an injury or infection, the pressure increase may not be chronic or permanent, but it may last for days or weeks, which is more than long enough to inflict severe and permanent damage to the retina. Therefore, the ability of carotenoids to help reduce and control inflammation, even if that beneficial activity is only mild and weak when compared to potent drugs such as steroids, may be extremely helpful and crucially important in protecting eyes and vision against permanent damage caused by decreased retinal blood flow, caused by inflammation due to an injury or infection.

It must also be recognized that anti-inflammatory steroids cannot be given to patients for long periods of time, without causing serious side effects (as can be observed in patients who must take steroids for extended periods, due to diseases such as lupus). Therefore, even though anti-inflammatory steroids are highly useful for treating acute inflammation following an infection or injury, they are not useful or desirable for most types of long-term use. By contrast, carotenoids can and should be a part of a normal and healthy daily diet for a person's entire lifetime.

(2) Carotenoids also have mild yet potentially helpful ability to prevent and reduce sclerosis, as described in articles such as Carpenter et al 1997. “Sclerosis” refers to hardening, stiffening, and loss of flexibility (for example, arteriosclerosis refers to hardening of the arteries, while atherosclerosis is a related process in which the insides of the arteries become coated with cholesterol or other fatty deposits).

In the eyes, sclerosis and loss of flexibility can arise not just in blood vessels, but also in certain layers and structures of the eye, especially if substantial quantities of drusen, lipofuscin, and other debris accumulate in those layers and structures. In addition to rendering the eye less able to focus on objects at varying distances, this loss of flexibility can damage certain membranes, such as the Bruch's membrane, a crucially important layer behind the retina. Therefore, by helping prevent and reduce sclerosis, even if only mildly, carotenoids can help protect eye health and good vision.

(3) Carotenoids also have mild yet potentially useful levels of activity in controlling and regulating angiogenesis (i.e., the formation of new blood vessels), because of their ability to help suppress various angiogenic hormones and cytokines, as described in articles such as Armstrong et al 1998 and Chew et al 2003. Since the formation of new blood vessels can lead to severe problems and blindness in “wet” or “exudative” macular degeneration, this activity of carotenoids may offer significant advantages not just in treating wet macular degeneration, but in preventing it in the first place.

(4) As described in articles such as Chew et al 2003, carotenoids have mild yet potentially useful levels of activity in helping to support and stabilize mitochondria and suppress cell death due to apoptosis.

(5) Carotenoids have mild yet potentially useful levels of activity in helping regulate and control certain types of actions and responses of the immune system, as described in articles such as Walrand et al 2004 and Carpenter et al 1997. These activities may be manifested in ways that relate to inflammation, suppression of dendritic killer cells that otherwise might carry out apoptosis too soon, etc.; however, this type of mild and subtle contribution to proper regulation of the immune system can also be manifested in other potentially useful ways, as well. Accordingly, this factor should be noted, and kept in mind. It must also be noted that the five “secondary” activities of carotenoids, listed above, also act in addition to the two primary activities of carotenoids, which are (1) protection against destructive ultraviolet radiation, and (2) protection against destructive oxygen radicals).

Upon recognizing that carotenoids (a class of highly specific compounds that cannot even be created by animals, and which must be ingested in animal diets) play at least seven different useful activities, in animals, the inventors began looking deeper into underlying factors and activities. The realizations that were gradually reached fit into a larger framework of study and understanding involving eye and vision disorders. Several factors and insights that can help describe and explain that framework, and that help show how that framework can be put to good use, focus on “connecting rods” that connect different parts of the frame to each other. Four of those “connecting rods” can be summarized as follows:
(i) oxygen radicals play roles in (or are created in increased quantities by) several different classes of problems, which may manifest in different but overlapping ways, such as in tissue inflammation and immune responses; it should also be noted that elevated quantities of oxygen radicals can trigger the production of inflammatory cytokines, as described in articles such as Ohgami et al 2003, Lee et al 2003, and Armstrong et al 1998, and Ford et al 2003; and, elevated quantities of oxygen radicals are also produced by certain types of immune cells, which use those oxygen radicals to help kill and digest microbes, as described in articles such as Walrand et al 2004;
(ii) mitochondria are also deeply and heavily involved in numerous processes that use or manipulate oxygen, and that can generate oxygen radicals;
(iii) cells have only limited numbers of signaling pathways they can use to communicate with each other; and,
(iv) reports have indicated that people suffering from various types of eye problems also suffer from low carotenoid concentrations in their blood (as shown by tests on blood serum), and in their eyes (as shown by low levels of macular pigment, and low concentrations of zeaxanthin in the lenses of people suffering from cataracts);

Accordingly, after realizing that carotenoids may be called upon to perform a number of different secondary activities in addition to two main primary activities, the inventors reached two important conclusions about carotenoids in human health, and especially in the eyes. Those conclusions can be summarized as follows:
1. If carotenoids are being asked to perform at least seven different known tasks (and possibly even more), all of which can converge and rise to levels of major importance if and when the eyes begin to suffer from serious problems, then carotenoids are more likely than other types of molecules to become “stretched thin”, to a point where their concentrations will drop and they will not be able to adequately handle all of the tasks and problems that are being pushed at them;
2. If any of the “secondary demands” that may tend to “siphon off” the desired concentrations of carotenoids in the eyes can be reduced, by means such as administering other nutrients that can provide a balanced regimen that will help address and satisfy those secondary demands, then any newly-arriving carotenoids will be more likely to actually arrive at locations where they can carry out their essential primary roles and provide the most overall benefit.

Viewed from another perspective, zeaxanthin can be regarded as a form of “buffer”, in a system that is constantly trying to sustain an equilibrium, or “homeostasis”. Like other types of buffer compounds, carotenoids can respond to whatever is added to (or imposed upon) the system, in a way that usually will help the system move back toward its equilibrium (also referred to as the “set-point” of the system). However, if the outer limits and maximum capacity of a buffering system has been reached, addition of even a slight quantity of additional stress (such as an acid or alkali) can cause major swings and upheavals.

Accordingly, if the “buffering system” provided by carotenoids in a human eye has already been stretched to its limit by a combination of multiple competing demands, the protective “buffering system” can fail, leading to a series, cascade, or mixture of stresses, problems, and damage, all occurring at once and all acting together, in ways that are suggested by phrases such as vicious circle, witch's brew, etc.

Accordingly, it is believed that this entire set of problems can be addressed by certain combinations, including optimized combinations of both zinc and zeaxanthin, both of which can function, in effect, as buffering systems.

Despite the strong preference for zeaxanthin for use in formulations as disclosed herein, lutein is covered by any claims below that refer to “macular pigment”. Although it is believed by the coinventors herein that zeaxanthin can and will provide better results than lutein in preventing and treating macular degeneration and possibly also other eye or neurodegenerative diseases, it should be recognized that certain large companies are making large profits from lutein, and they want to continue doing so. Accordingly, several such companies are acting in ways that clearly indicate that they regard zeaxanthin as a threat to their profits, rather than as a better way to help prevent blindness or neural degeneration. This is clearly manifested in the current plans (as of May 2005) for the AREDS-2 trial, in which a large and powerful company has pushed forward and advanced proposals that would test only a single carotenoid formulation with a lutein dosage five times higher than zeaxanthin, even though experts in the field have been informed that zeaxanthin has a better chemical structure and is at the center of the macula, while lutein is relegated to the periphery of the macula. In addition, the current plans for the AREDS-2 trial would block anyone from being able to analyze, evaluate, or quantify the differing contributions of zeaxanthin versus lutein in protecting eye health, even though the available facts clearly indicate that the human macula: (i) wants and needs zeaxanthin, (ii) uses lutein because it cannot obtain enough zeaxanthin, and (iii) even tries to convert lutein into zeaxanthin.

Accordingly, inclusion of lutein in the “macular pigment” claims herein is intended to help create and promote a situation in which actual and lasting benefits for the eyes, vision, and brains of elderly consumers, will take priority over profits for large companies. Allowing large companies to avoid a set of patent claims and make higher profits, by substituting lutein for zeaxanthin in their products even though lutein does not work as well as zeaxanthin, would be counterproductive from the viewpoint of actually helping the public health and welfare (especially when it comes to helping grandparents get to see their grandchildren grow up). Accordingly, lutein is covered by various claims below, not because it is equal to or interchangeable with zeaxanthin (it isn't), but to help ensure that the eye care and nutritional supplements industries are constrained and obliged to give elderly consumers the best help (and the best research) that can be provided, in the struggle against a terrible disease that often leads to blindness among the elderly.

Two additional points should be noted. First, it is believed that treatment with zeaxanthin, at any reasonable dosages of interest, is free of any significant risks, among people who are suffering from macular degeneration or other eye problems. That statement is supported by a “New Dietary Ingredient” application that was filed on zeaxanthin, with the U.S. Food and Drug Administration, by Roche Vitamins, Inc. That NDI application contained extensive safety data, including data from animal tests indicating that even at very high dosages, zeaxanthin did not cause any pathological changes, of any sort, in animal tests. The docket number of that NDI application, published in June 2001, is 95S-0316. It can be downloaded at no charge from the FDA website.

Second: this is a very low-cost treatment, compared to the costs of either macular degeneration (which often leads to functional blindness), or verteporfin-laser (photodynamic) treatments for the minority of patients who suffer from “wet” macular degeneration. For example, each verteporfin-laser treatment costs thousands of dollars. By contrast, 60 capsules of zeaxanthin, containing 10 mg each, cost roughly $60, from www.zeavision.com.

Meso-Zeaxanthin and Zeaxanthin Esters

The dietary 3R,3′R isomer of zeaxanthin (also referred to as the R,R isomer, for convenience) is strongly preferred for use herein, over the non-dietary meso-zeaxanthin isomer. However, certain companies (mainly companies outside the United States, who are not governed by the laws that apply in the U.S.), have begun advertising “zeaxanthin” that was created by chemical treatment of lutein, apparently without warning prospective buyers that the “zeaxanthin” they are selling is actually a non-dietary isomer that may be violating US laws if imported into the United States. Therefore, an explanation of the differences between those two stereoisomers becomes necessary.

Except for very small amounts (measured in nanograms) that can be found in human retinas as a result of the lutein conversion process mentioned above, meso-zeaxanthin has never been found in nature, or in any plant or food sources. This assertion requires an explanation and defense, since it contradicts a claim that was published in 1986 by researchers working in Japan. Maoka et al 1986 asserted that meso-zeaxanthin had been found in certain types of marine life, such as in the skins of certain types of fish. However, that claim was directly contradicted and undercut, years later, by Khachik et al 2002, which showed that the type of chemical processing used by Maoka et al caused lutein to be converted into meso-zeaxanthin (information on how that type of chemical processing converts lutein into the meso-zeaxanthin isomer is available in Bone et al 1993).

The problems with Maoka et al 1986 become clear from a careful analysis of page 3388 (column 2) of Khachik et al 2002, which reported that when the same processing steps that had been used by Maoka et al were used by Khachik et al, meso-zeaxanthin appeared to be present in human blood. However, when more modern and accurate processing methods were used by Khachik et al, meso-zeaxanthin was shown to be completely absent from human blood. In other words, Khachik et al 2002 directly showed that the type of chemical processing used by Maoka et al in the 1980's could and would create meso-zeaxanthin, when carried out on a biological liquid.

Therefore, no one can accurately or reliably claim that meso-zeaxanthin has been found in any dietary source. Any such claims that are based on Maoka et al 1986 are misleading and unsupported, because the findings in Maoka et al 1986 were directly contradicted by subsequent research using better and more accurate methods.

Since meso-zeaxanthin has never been found in human blood, there is a consensus that any meso-zeaxanthin found in human maculas must be formed by lutein conversion. Accordingly, the fact that the human macula does indeed try to convert lutein into zeaxanthin (resulting in a stereoisomer that does not otherwise exist in nature) is strong evidence that the human eye “prefers” zeaxanthin over lutein.

Because of its close similarities to R,R-zeaxanthin, meso-zeaxanthin (the nondietary S,R isomer) may be able to provide some protective benefits, if ingested in a nutritional supplement, instead of or in addition to normal dietary R,R-zeaxanthin. Accordingly, all references to “zeaxanthin” in the claims are specifically intended to include meso-zeaxanthin. However, any supplier, optometrist, or ophthalmologist who might consider selling, using, or prescribing meso-zeaxanthin in humans should be aware of the following concerns and issues:

1. As summarized above, the claim that meso-zeaxanthin has been found in certain types of marine life has been contradicted and undercut. To the best of the coinventors' knowledge and belief, meso-zeaxanthin has never been found in any normal and natural dietary sources; instead, the meso-zeaxanthin that has recently been offered for sale, by certain companies operating outside the United States, is being made by harshly alkaline treatment of lutein, from marigold flowers.

2. Since meso-zeaxanthin is a non-dietary isomer, as stated in numerous published articles, and since it apparently is being manufactured by chemical treatment of lutein, then the statutory language in the Dietary Supplement Health and Education Act (the DSHEA statute, passed by Congress in 1994) requires certain legal requirements to be met before it can be sold or administered to humans in the U.S. In particular, the language concerning “chemically altered” food compounds, in section 413 of the DSHEA act (codified in 21 U.S. Code 350b) needs to be evaluated carefully, since it sets forth various legal requirements that must be met before any such chemically-altered food can be sold legally, in the U.S.

3. Recent searches of publicly accessible records on the U.S. Food and Drug Administration database indicate that the legal requirements of 21 USC 350b have not been met by any companies in the U.S. that are buying or selling meso-zeaxanthin. As of this writing, it appears that no regulatory filings have been made, by any seller, supplier, or distributor of meso-zeaxanthin, of the type that would be necessary to enable legal and lawful sales of meso-zeaxanthin to humans in the United States, or to companies that might put it into the human food chain (such as by feeding it to poultry or farm-raised salmon).

4. When a test was done in Spain to compare meso-zeaxanthin against natural R,R-zeaxanthin as a color additive for poultry, the meso-zeaxanthin preparation did not perform satisfactorily. The results are described in Perez-Vendrell et al 2001. In addition, based on a website posting by Texas A&M University, describing an annual conference on poultry feeds and pigments, supplemented by inquiries by an industry consultant, it is believed that a similar test was done in Mexico, and the results of those tests also showed unsatisfactory pigmenting results for meso-zeaxanthin, in poultry. Both of those two sets of results have indicated that meso-zeaxanthin apparently is not deposited into animal tissues in a manner that closely follows the normal patterns of deposition that occur with natural R,R-zeaxanthin.

It also must be recognized that stereoisomers of biomolecules can be crucially important. In the field of nutrients, stereoisomer configurations of numerous important compounds (notably including sugars and amino acids) are crucially important, and the wrong isomers can be toxic or even deadly. In addition, it has been recognized that differing stereoisomers of various drugs can have very different effects, toxicities, and metabolic fates, to a point where the Food and Drug Administration has adopted a formal policy on drug stereoisomers. That policy requires drug companies to specifically attempt to synthesize, purify, and tests the effects of different stereoisomers of any proposed new drug, before any such drugs are tested in humans. The company must compile, and must submit to the FDA, any data that are observed on how the different stereoisomers perform.

In view of all of these factors, R,R-zeaxanthin (as found in the diet) is strongly preferred over meso-zeaxanthin (the nondietary stereoisomer). However, since foreign suppliers are sending unapproved and potentially illegal supplies of meso-zeaxanthin into the United States, under labeling and advertising that calls their products “zeaxanthin” while hiding the fact that they are selling a nondietary and potentially dangerous version, all references to “zeaxanthin” in the claims include meso-zeaxanthin. A patent allows the patent owner to stop others from making, using, or selling a patented invention without permission; accordingly, coverage of meso-zeaxanthin by the claims is intended to help protect the public against an unapproved and possibly illegal compound that apparently failed when tested in animals.

In addition, any references herein to “zeaxanthin” include esters of zeaxanthin. Most plants and some bacteria synthesize lutein and zeaxanthin, not in the form of “free” carotenoids (also called “alcohol” carotenoids), but with one or two fatty acids linked to the hydroxy groups in a manner that creates an ester bond. However, when these esters are ingested by animals, most of the ester linkages are broken, in a manner that releases free (non-esterified) zeaxanthin or lutein. This chemical reaction is often called “hydrolysis”, since a water molecule is effectively inserted into what was previously the ester bond. These reactions are catalyzed by esterase enzymes and certain other digestive enzymes. Accordingly, since zeaxanthin or lutein esters will release “free” zeaxanthin or lutein after ingestion by an animal, they are regarded as nutritionally equivalent, and any reference herein to any carotenoid that contains a hydroxy group also includes the ester form.

Neuroprotective Agents that can Reduce Zinc Risks

If desired, any zinc-containing formulation as discussed herein can also contain one or more active agents of a type referred to herein as “neuroprotective” agents, to help reduce one or more of the neurologic risks that are created or increased by high levels of zinc in the brains of elderly patients. Candidate neuroprotective agents that merit consideration are briefly listed below, with citations to article on their roles and effects. Additional information on these compounds is also provided in Patent Cooperation Treaty application PCT/US04/43743, by Gierhart (one of the inventors herein), which will be published in about June 2005.

1. Agents that boost mitochondrial functioning can help prevent cells from dying due to a process called “apoptosis” (described in reviews such as Ravagnan et al 2002, van Gurp et al 2003, and Mayer et al 2003). Such agents include Coenzyme Q10 (e.g., Menke et al 2003), omega-3 fatty acids (e.g., Connor 2000, Jeffrey et al 2001, Polit et al 2001, Murayama et al 2002, and Rotstein et al 2003), taurine (e.g., Devamanoharan et al 1998, Obrosova et al 1999 and 2001, Chen et al 2000, Militante et al 2002, Pasantes-Morales et al 2002, and DiLeo et al 2003), creatine (Tarnopolsky et al 2001), and carnitine (e.g., Pessotto et al 1997, Peluso et al 2001, Alagoz et al 2002, and Feher et al 2003). “Glutathione boosters”, discussed below, can also function as mitochondrial boosters.

“Mitichondrial medicine” is also discussed in Tarnopolsky et al 2001, Fosslien 2001 and 2003, Mahoney et al 2002, Zeviani et al 2003, and Rego et al 2003. In vitro assays can use toxins such as rotenone, malonate, or 3-nitropropionic acid to impair mitochondrial functioning, and compounds that can help stabilize mitochondria in such “challenge assays” include rasagiline (e.g., Youdim et al 2003) and idebenone (e.g., Mahoney et al 2002).

A combination product sold under the name PHOTOTROP™ by the Sigma Tau Company contains three mitochondrial-boosting agents listed above (Coenzyme Q10, carnitine in the form of a precursor, acetyl-L-carnitine, and omega-3 fatty acids), sold in the form of orally-ingested capsules. However, those capsules do not contain any zinc, Vitamins C or E, or zeaxanthin or other carotenoids. Therefore, that formulation is regarded herein as a useful adjunctive treatment, for elderly consumers who take a “modified AREDS formulation” as disclosed herein, containing Vitamins C and E, zinc at a reduced dosage as disclosed herein, and zeaxanthin or an alternate neuroprotective agent.

2. Glutathione is a tri-peptide, formed by three amino acids with cysteine in the middle. The cysteine residue has a reactive sulfur group (—SH, also called a mercaptan, sulfhydryl, or sulfide group), which allows glutathione to bond to other compounds. This bonding makes waste metabolites more soluble in water, and it helps cells and tissues eliminate wastes, through pathways that lead to urine excretion. Glutathione can also help boost mitochondrial functioning. For these reasons, “glutathione boosters” can help stabilize mitochondria, suppress cell death through apoptosis, and protect eye health. Glutathione boosters include cysteine (and a precursor ester, N-acetyl cysteine) selenium, pyridoxine, and riboflavin, as discussed in U.S. Pat. No. 5,075,116 (LaHaye et al 1991) and U.S. Pat. No. 5,596,011 (Repine et al 1997). Analogs of glutathione may also merit attention for such use, such as S-nitrosoglutathione (Khan et al 2005).

3. Carnosine (also known as β-alanyl-histidine) is a di-peptide, formed in animal tissues when alanine and histidine bond to each other. It can quench and neutralize aldehydes, which otherwise can cause random damage to proteins. Since carnosine is a dipeptide, it is subject to relatively high rates of digestion and cleavage by protease enzymes, which will inactivate it. Therefore, its main ocular use to date has been in the form of eyedrops, which are sold in Europe. The benefits of carnosine, in eyedrops, are described in articles such as Maichuk et al 1997, Hipkiss et al 1998, and Babizhayev et al 2002.

In a recent development that becomes potentially important in the context of this invention, carnosine was tested to evaluate its ability to reduce the neurotoxic effects of zinc and copper, by in vitro cell culture tests using glial cells (i.e., brain cells that are not neurons and cannot receive or send out nerve impulses). The results, described in Horning et al 2000, led to the following conclusion: “Recently, we demonstrated that carnosine, a dipeptide expressed in glial cells throughout the brain as well as in neuronal pathways of the visual and olfactory systems, can modulate the effects of zinc and copper on neuronal excitability. This result led us to hypothesize that carnosine may modulate the neurotoxic effects of zinc and copper as well. Our results demonstrate that carnosine can rescue neurons from zinc- and copper-mediated neurotoxicity and suggest that one function of carnosine may be as an endogenous neuroprotective agent.”

Accordingly, if carnosine (or a precursor of carnosine) can be administered in ways or dosages that avoid or overcome the digestion and cleavage problems, resulting in significant concentrations in circulating blood, it offers a promising and preferred agent for use either as a component of, or as a treatment that can accompany, zinc-containing formulations for preventing or treating age-related eye disorders.

Oral administration of carnosine (or a precursor) in ways or dosages that will generate significant increases in blood-borne levels may be achievable, in one or more ways. In general, no cleavage reaction that occurs in digestion is ever 100% effective; therefore, dose-response tests that evaluate various levels of carnosine, in ingestible formulations that also contain other ingredients, should be considered, especially in combination with microencapsulation using keratin or other coating compounds to protect the carnosine from stomach acidity. In addition, precursors such as N-alpha-acetylcarnosine (an ester precursor that is cleaved by esterase enzymes to release carnosine) are known, and merit evaluation.

4. Agents that can stimulate enzymes called methionine sulfoxide reductases also merit attention. Methionine, one of the amino acids used to make proteins, can be present in cells and mitochondria either in oxidized form, or in reduced form. Excess quantities of the oxidized form can cause various problems, as described in articles such as Stadtman et al 2003 and 2005; and, oxidized methionine residues in beta-amyloid proteins may play a role in the release of beta-amyloid proteins from cell membranes, leading to the formation of beta-amyloid plaques in people who suffer from Alzheimer's disease (Barnham et al 2003). Therefore, methionine reductase enzymes can help protect cells, by returning methionine back to its reduced state, as described in articles such as Moskovitz 2005 and Yermolaieva et al 2004. Various compounds such as sulforaphane, thioredoxin, oltipraz, 1,2-dithiole-3-thione, and tert-butylhydroquinone appear to provide cell protective benefits, by means that apparently include stimulating the activity of methionine sulfoxide reductase enzymes, as described in articles such as Gao et al 2001, Morimitsu et al 2002, Kraft et al 2004, Konwinski et al 2004, and Tanito et al 2005. Accordingly, those and other compounds that can stimulate methionine reductase enzymes merit evaluation for inclusion in neuroprotective products as described herein for preventing or treating macular degeneration.

5. An additional set of candidate agents that deserve evaluation for use as described herein includes plant-derived compounds referred to as flavonoids (or bioflavonoids), isoflavones, anthocyanins, plant polyphenolics, phytonutrients, and phytohormones. These terms overlap heavily with each other, and compounds that fall within these categories are described in articles such as Beecher 1999 and Beecher 2003, and on websites such as http://www.friedli.com/herbs/phytochem/flavonoids.html.

Compounds within this category can include either or both of the following: (i) non-purified or semi-purified “extracts”, which typically are multi-component mixtures that have been extracted from the fruits, leaves, seeds, nuts, or other parts of certain plants such as bilberry, grapeseed, green tea, or soybeans; or, (ii) specific known and purified compounds (or limited mixtures of a small number of similar and related compounds), such as quercetin, genistein, diazedem, diadzin, glycitein, fisetin, luteolin, resveretrol, and pycogenol, and various known analogs of these compounds.

These active agents have different activities and roles, and each one needs to be considered separately. For example, some flavonoid compounds reduce the activity of an enzyme called aldose reductase, which converts beneficial sugars (such as glucose) into sugar-alcohols (such as sorbitol) that can cause problems if they accumulate (sorbitol is an important causative factor in cataract formation, especially among diabetics). Therefore, certain flavonoids can help prevent or reduce the formation of cataracts (e.g., Jung et al 2002, Matsuda et al 2002). Resveretrol reportedly can suppress vascularization (i.e., the formation of new blood vessels, as described in Brakenhielm et al 2001) and is a good antioxidant (Lorenz et al 2003), while genistein reportedly inhibits certain protein kinase enzymes, and can help suppress unwanted cell-signalling pathways (e.g., Yoon 2000). The specific activities of numerous plant polyphenols can be identified fairly easily, by searching the database maintained by the National Library of Medicine. Additional information is available from U.S. Pat. No. 5,952,374 (Clarkson et al 1999), which reports that certain flavonoids from soybeans can stimulate expression of certain genes inside the brain that can help fight Alzheimer's disease or other dementias.

Vitamins C and E, and Other Candidate Active Agents

Vitamins C and E preferably should be included in any antioxidant-plus-zinc formulations disclosed herein. The dosage ranges tested in the AREDS-1 trial are generally believed to be suitable, as described in U.S. Pat. No. 6,660,297 (Bartels et al 2003). However, in view of recent reports on cardiovascular risks associated with Vitamin E at high dosages, preferred daily dosages (either as alpha-tocopherol, or as other or “mixed” tocopherols) generally should range from about 100 to about 500 international units.

Since Vitamin C is water-soluble, it is secreted in urine if overconsumed, and there are no specific upper limits to safe dosages of Vitamin C, among the general public. However, as noted above, some reports have indicated that post-menopausal women who suffer from coronary artery disease should not take high dosages of vitamin C. Since nearly all women who are taking supplements to prevent or treat macular degeneration will be post-menopausal, and since nearly anyone who has reached his or her 60's or 70's will have at least some plaque buildup inside their arteries by that age, dosages of Vitamin C in formulations as described herein should be limited to a moderate range, such as a range of about 100 to about 500 mg/day.

As mentioned above, the best and most reliable assessments of truly optimal dosages of relatively safe and well-known, widely-used agents (such as Vitamins C or E), in formulations such as described herein, can and should arise from tests of formulations that also include at least 5 and preferably at least 10 mg/day of zeaxanthin. Most macular degeneration cases may well arise from a straightforward vitamin deficiency, and zeaxanthin appears to be the vitamin sitting at the center of that problem, just as it sits in the center of the macula.

Vitamin A (normally ingested in the form of its precursor, β-carotene) can be included in reduced-zinc formulations as disclosed herein, if desired. However, for several reasons, β-carotene preferably should be either omitted entirely, or it should be limited to relatively low dosages, such as found in multi-vitamin formulations (such as a daily dosage range of about 2500 to about 5000 international units (IU) per day, which is about 0.5 to 1 RDA, and which is much less than the 28,640 IU dosage in OCUVITE PRESERVISION or ICAPS. This reasons for this include the following: (i) β-carotene can compete against zeaxanthin for digestive uptake into circulating blood; (ii) while β-carotene is a useful anti-oxidant at low oxygen levels, it can turn into a damaging pro-oxidant at high oxygen levels that may exist in some tissues, including lung tissues; and, (iii) in large and well-run clinical studies, it was shown to increase the risks and rates of lung cancer among smokers, and there is a large and heavy overlap between smokers and macular degeneration sufferers, since smoking is one of the known risk factors that directly increases rates of macular degeneration.

In addition to the various agents listed above, other known anti-oxidants that were not tested in the AREDS-1 trial also merit consideration, for inclusion in an ocular formulation that can help prevent or treat macular degeneration, cataracts, or other eye or vision disorders.

One such candidate anti-oxidant is selenium, the mineral. In particular, it may be able to help supplement the anti-oxidant activities of a reduced dosage of zinc, thereby making up (at least partially) for the reduced dosages of zinc in the formulations described herein, compared to the AREDS products.

Another candidate anti-oxidant that merits attention is lipoic acid, a fatty acid that alternates back and forth between a reduced form, and an oxidized form. Lipoic acid can help reduce and prevent unwanted oxidation of cells and tissues; under some circumstances, it also can help regenerate vitamin E (Stoyanovsky et al 1995). Other articles that describe lipoic acid's ability to protect ocular tissues in various tests include Packer 1994, Obrosova et al 1998, Borenshtein et al 2001, Chidlow 2002, and Goralska et al 2003. Maitra et al 1996 reported that the naturally-occurring “R” (dextrorotatory) stereoisomer has better anti-oxidant activity than the S (levorotatory) isomers that are found in synthetic racemic mixtures.

It is believed that a well-balanced multi-component formulation containing zeaxanthin along with one, two, or more of the agents discussed above will be able to significantly outperform the formulation tested in the AREDS-1 trial, to an extent that will help consumers and physicians realize that inclusion of a heavy dosage of zinc simply is not necessary to obtain good results.

Preventive and Therapeutic Dosages and Formulations

Preferred formulations for use herein include convenient orally-ingestible forms, which may be in any of three general forms: (i) unit dosage forms, such as capsules or tablets; (ii) fortified foods or foodstuffs containing additives as described herein; or, (iii) beverages (or beverage ingredients, such as mixable powders, syrups, etc.), which can be regarded, labeled, and used either as foodstuffs, or as unit dosage formulations.

Any of these orally-ingestible formulations can contain relatively high dosages of active agents for therapeutic treatment of people who have been diagnosed as having macular degeneration, lens clouding, or other ocular disorder(s), or who have noticed that they are suffering from a vision problem. Alternately, such formulations can contain somewhat lower dosages, which can be referred to as preventive, maintenance, or prophylactic dosages (or similar terms), for sustaining generally good eye health rather than for treating a diagnosed or other known problem. Any such dosages (either at therapeutic or preventive levels) can be determined and expressed either as unit dosages (typically expressed as the number of milligrams of each active agent in each tablet, capsule, or other dosage unit), or as daily dosages (usually determined by multiplying a unit dosage per tablet or capsule, times the number of tablets or capsules recommended for ingestion each day).

There is no sharp dividing line between therapeutic dosages and preventive dosages, and they may overlap. For example, a person may realize that his or her vision has become blurred, either because one eye can no longer see as clearly as the other, or because he or she takes a non-medical vision test, such as one seen in a magazine or administered at a driver's license office. A person with that type of self-diagnosed or suspected problem may choose to take either a supplement having a low to moderate dosage for preventive purposes, or a higher therapeutic dosage for treatment purposes. Because most formulations as disclosed herein will contain only agents that can be sold over-the-counter without a doctor's prescription, the choice of dosages (and the number of dosages taken per day) will be a matter of personal preference.

It should be noted that a single unit dosage formulation can be used to obtain either higher therapeutic dosages or lower preventive dosages, merely by controlling the number of unit dosages ingested each day. For example, capsules containing preventive dosages can be ingested once daily, by consumers who do not suffer from eye problems and who want to take appropriate nutritional supplements that can help sustain good vision. The same capsules with the same unit dosages can be taken two or three times daily, by people who are suffering from a diagnosed or clearly evident problem and who need therapy to try to reverse or at least slow down the decline.

Similarly, when a person first begins to take these supplements, he or she may choose to take two or three capsules per day during the first month or two, in order to more rapidly reach and attain desired levels of these agents in circulating blood, and then reduce his or her daily dosage to a single capsule per day, after the first or second month. Indeed, because one of the main aspects of the invention herein centers on an effort to appropriately balance the benefits of zinc (in preventing or treating macular degeneration) against the neurologic risks of high-dosage zinc among the elderly, a preferred and recommended dosage regimen for use herein will involve two stages. The first stage will be an initial accelerated or “buildup” regimen, designed to establish higher blood concentrations, by daily ingestion of two to four capsules or other unit dosages each day, for roughly 4 to 8 weeks after a person begins taking them. When the initial stage is completed, it will be followed by long-term sustenance regimen, involving reduced daily dosages, such as only one or possibly two capsules per day.

As mentioned above, in the Summary section, now that the conflict at the heart of this invention (i.e., high zinc dosages are good for aging eyes, but dangerous for aging brains) has been identified and focused upon, it is hoped that this disclosure will lead to additional attention to this issue, by researchers affiliated with academia, medical schools, government agencies, and not-for-profit foundations. It is also hoped that their efforts will lead to treatments that can and will take the disclosures herein as a baseline that can be improved upon, by identifying and evaluating additional active agents (including various agents as listed above), which may include drugs that at the current time are available by prescription only (or that are limited to small-scale human clinical trials), but that would deserve approval for over-the-counter sale and use, if they are shown to substantially reduce the neurotoxic risks of high-dosage zinc ingestion among elderly consumers.

Animal Models of Macular Degeneration and Protection

Anyone working in this field should be aware of various types of in vivo animal models and human clinical trials that can be used to evaluate both the macular protective and neuroprotective aspects of the formulations disclosed herein.

At least five different and distinct animal models are known for testing candidate ocular-active nutrients. These models include the following:

1. Mice and Rats, Including “Knockout” Mice

Mice and rats are widely used in research on small animals, and a huge foundation of information, species-specific biomolecules (including gene promoter sequences, gene coding sequences, monoclonal antibodies, etc.) and specialized strains have been developed for genetic work with mice. Gateways that can be used to access mouse genetic information are freely available on websites such as www.informatics.jax.org and www.ncbi.nlm.nih.gov/genome/seq/MmHome.html. Although the corresponding genetic information on rats is somewhat smaller, it is still enormous and quite useful, and can be accessed through websites such as http://rgd.mcw.edu, http://ratmap.gen.gu.se, and www.hgsc.bcm.tmc.edu/projects/rat.

This genetic information can be put to good use, because a growing number of gene defects have been and are being correlated with known eye disorders. These genes can be discovered by any of several procedures. For example, research revealed that many people who suffer from Stargardt's disease, which causes severe vision impairment have a defective protein known as the Rim protein, which normally functions as an ATP-binding cassette (ABC) transporter gene, in rod outer segment discs, in mammalian retinas. Additional research on that protein (and the gene which encodes that protein) led to identification of a gene called the ABCR gene, as the specific defect that leads to the defective protein in people who suffer from Stargardt's disease. If no properly functioning copies of the ABCR enzyme are present, a toxic metabolite called A2E (shown and described in Radu et al 2003) will gradually accumulate over a span of years in the retinal pigmented epithelial (RPE) layer, directly behind the retina. This accumulating toxin will eventually cause severe damage to the RPE, which is crucial for supporting the retina. As a result, Stargardt's disease usually leads to functional blindness by the time a person reaches the age of about 20. The most common form of Stargardt's disease is a recessive disorder, which normally occurs only in people who have inherited dysfunctional ABCR genes from both parents (this is referred to as the ABCR −/− genotype). If a person has inherited a properly functioning gene from one parent, and a dysfunctional ABCR gene from the other parent (referred to as the ABCR +/− genotype), Stargardt's disease will not be fully manifested; however, the person will be at elevated risk of serious eye and vision problems later in life.

After the human ABCR gene was identified as a causative factor in Stargardt's disease, a “homologous” ABCR gene in mice was located, which encodes the mouse version of the rim protein. The exact DNA sequence of the mouse ABCR gene was determined, and researchers then used genetic engineering techniques to create mutant mice with “knockout” ABCR genes that are no longer properly functional. These mutant mice, with “knockout” ABCR genes and the mouse equivalent of Stargardt's disease, are described in articles such as Weng et al 1999, Mata et al 2000, Radu et al 2003, and Radu et al 2004. Their descendants suffer from severe visual impairment, which grows gradually worse as certain waste metabolites gradually accumulate within their retinas and retinal pigmented epithelium (RPE) layers. Therefore, the descendants of these knockout mice offer useful animal models, for testing candidate nutrients that may be able to help slow down the gradual accumulation of toxic metabolites in people who have ABCR −/− genotypes (leading to Stargardt's disease) or ABCR +/− genotypes (leading to vision problems later in life).

Two additional mouse models of eye diseases that are worth noting include mice that have defective or missing genes that encode proteins referred to as monocyte chemoattractant protein-1 (Ccl-2, also known as MCP-1) and its cognate C—C chemokine receptor-2 (Ccr-2), as discussed in Ambati et al 2003. When those genes are defective, the infiltration of macrophages (specialized types of immune cells) into certain retinal structures (such as the choroidal membrane) is altered, in ways that lead to pathologies that emulate age-related macular degeneration in those retinal tissues.

Yet another mouse model is provided by mice that have defective or missing genes that encode apolipoprotein-E, which is essential for making a properly-functioning Bruch's membrane behind the retina, as discussed in articles such as Dithmar et al 2000 and 2001 and Miceli et al 2000.

These are three specific examples of known gene defects that lead to retinal pathologies, and each of these defects has been used to create an animal model that can be used to test and evaluate the potency of various candidate agents that may be able to help prevent or treat macular degeneration or other retinal pathologies in humans. Other useful animal models are being developed and published by other research teams, and additional information is available in review articles such as Ambati et al 2003.

This work is being expanded and accelerated greatly by various genetic engineering methods, and by using and comparing gene sequence information that already has been gathered and published as part of the human genome project, the mouse genome project, and the rat genome project. Dozens or even hundreds of genes that express specific proteins involved in eye structures and/or vision processing have been identified, and each such discovery involving a specific gene provides a potentially useful new and distinct animal model for testing.

Four presumptions apply to such research: (1) every structural protein that is present in any eye structure, and every enzymatic protein that is involved in any step in vision processing in the eyes, is present within the eyes for a good reason, and plays some useful and necessary role in vision; (2) a gene defect that renders any such protein nonfunctional will very likely lead to some type of identifiable and potentially important eye disorder; (3) once any such genetic defect has been identified, either in humans or in mice or rats, colonies of lab animals that carry that genetic defect can be created and/or raised; and, (4) any such colony can provide an animal model that can help researchers evaluate and rank the ability of various candidate nutrients or other treatments to overcome the problem that is caused or aggravated by that particular defective gene or protein, in that animal model.

Accordingly, genetic analysis and research, including research involving mice or rat colonies having “knockout” genes that are correlated with specific vision disorders, offer extremely powerful tools, and can provide an effectively unlimited number and range of specific targeted “models” that can help researchers test candidate nutrients, to evaluate whether any nutrient or nutrient combination can act synergistically with zeaxanthin, to help prevent or treat one or more specific types of ocular disorders.

2. Use of Agents to Increase Carotenoid Uptake in Rodents

When carrying out vision-related research on mice or rats, it must be noted that most rodents are prey rather than predators, and they rarely go out into direct sunlight during the day, since that would make them highly vulnerable to predators. Accordingly, rodents did not evolve with any need for carotenoids to help protect them against UV radiation. Therefore, rodents generally do not metabolize carotenoids in ways comparable to humans, and they tend to make relatively poor models for studying the uptake or effects of carotenoids.

However, various manipulations can be used to increase carotenoid uptake in rats and other rodents. As one example, if relatively high concentrations of bile salts or other compounds that help solubilize fatty compounds are added to the diets of mice or rats, the animals will transport higher quantities of carotenoids through the intestinal walls and into circulating blood, which will lead to greater rates and concentrations of tissue deposition. Therefore, by feeding special diets to mice or rats, various types of research involving zeaxanthin (or other carotenoids) can be carried out in these animals.

It should also be recognized that research which directly uses and includes zeaxanthin will not always be necessary, to do research on mice or rats that can help evaluate and rank candidate nutrients that may be able to work synergistically with zeaxanthin. Instead, the benefits of working with mice or rats usually are limited to initial research, which hopefully will lead to expanded and more expensive research on larger animals and/or humans. Accordingly, mice and rats may be well-suited for evaluating candidate nutrients such as lipoic acid, isoflavonoids, plant polyphenols, omega-3 fatty acids, taurine, carnitine, etc., to evaluate their effects on ocular or vision defects, in tests that will not use or include any zeaxanthin or other carotenoids. Subsequently, after initial evaluations and rankings have been determined by means of initial testing in mice or rats, the most promising candidates can then be tested in more expensive tests that will involve zeaxanthin, using animals that metabolize carotenoids in a manner comparable to humans (such as Japanese quails or other suitable birds, or primates), or in human clinical trials.

It should also be recognized that mice, rats, and other rodents do not have pigmented maculas; instead, in general, the only animals that use UV-absorbing carotenoids to protect their retinas are primates, and some species of birds. However, if rats are induced (by bile salts in their diets) to begin taking up substantial quantities of carotenoids into circulating blood, at least some of those carotenoids will be deposited into photoreceptors in the retina, and into the lens of the eye, thereby allowing at least some types of research on those structures.

3. Agents and Methods to Create and Emulate Disorders

Additional options that can be used to evaluate candidate ocular-active nutrients involves the use of certain drugs or diets, to induce certain types of damage that can emulate known ocular disorders. As one example, cataracts can be induced by a drug called buthionin sulfoximine (e.g., Maitra et al 1996), or by feeding lab animals certain types of high-starch diets (e.g., Borenshtein et al 2001). As another example, diabetes can be induced by drugs such as streptozotocin (e.g., Kowluru et al 2003) or allosan.

If the goal of a research project is to study a disorder that involves abnormally high levels of cell growth (such as wet macular degeneration, with excessive blood vessel growth, or certain types of “proliferative retinopathies”), pellets contain cell-stimulating hormones can be implanted into an eye. Such research, using “vascular endothelial growth factor” (VEGF) or “basic fibroblast growth factor” (bFGF), is described in articles such as Yoon et al 2000 and Joussen et al 2000.

Various types of surgical or mechanical interventions can also be used to emulate certain ocular disorders. As one example, clamping off an artery for a fixed period of time is used to create ischemia, then the clamp can be suddenly released, to create a “reperfusion” injury involving oxygen free radicals. In addition, external methods can be used to accelerate certain types of visual impairment. Such methods include, for example, increasing the intensity of ultraviolet and blue light, and increasing the atmospheric oxygen concentrations, in the pens or rooms where lab animals are being kept.

Any of these methods can impose additional levels of ocular stress on lab animals, thereby substantially accelerating the rates at which they will develop ocular disorders. Accordingly, various candidate ocular-active nutrients can be evaluated for potency and efficacy, by measuring how effectively they can delay, prevent, or reduce the disorders that will arise from the stresses that were imposed on the animals.

4. Japanese Quail and Other Birds

As mentioned above, some types of birds use carotenoid pigments to help protect their retinas against damage by UV light. In most bird species, these pigments are deposited throughout the entire retina, rather than just in a small central area comparable to the maculas of primates. A review of the use of birds, in retinal research, is contained in Fite et al 1991. Japanese quail have become a widely used and accepted bird model for retinal testing, as described in articles such as Fite et al 1993, Fite 1994. Detailed methods for testing this species, to evaluate the ability of zeaxanthin or lutein to protect against retinal damage caused by high-intensity lights, were described in Thomson et al 2002.

In addition, an albino strain of Japanese quail has been developed, which suffers from rapid lens degeneration and cataract formation.

5. Testing of Dogs and Livestock

Among the types of lab animals larger than rodents that are used in vision testing, dogs and livestock tend to be used most commonly, for various reasons.

With respect to dogs, their irises (which are circular) are more similar to human and primate irises, than the vertical-slit irises of cats; in addition, dogs also suffer fairly commonly from cataracts. They can also be induced to incur various types of retinopathies, and there are certain aspects of their vision processing that are of interest to neurology researchers (including limitations in the ability of dogs to generate nerve impulses that will help them recognize and identify things, unless some type of motion is involved that will trigger a set of nerve cell firings). For all of these reasons, dogs are used fairly commonly for ocular and vision research. While they are more expensive than mice or rats, they are less expensive than primate studies or human clinical trials. Accordingly, if dogs are being considered as a potential animal model for studies as disclosed herein, a network of experts who are already familiar with that type of research in dogs can be located, quickly and easily, by a search for published articles describing vision research in dogs.

Research on eye components or other tissues from various livestock species (including pigs, cows, and sheep) is enabled by an important factor: these animals are killed, in large numbers, at known locations and under controlled conditions (i.e., at slaughterhouses). Therefore, specialized treatment procedures can be carried out on livestock animals shortly before they are killed, and the affected tissues can be harvested at a controlled time, soon thereafter. Alternately, other types of specialized procedures can be carried out on tissue that was harvested immediately after an animal is killed; these types of tissue samples are usually perfused (i.e., placed in specialized equipment that will pump fluids with oxygen and nutrients through or around the tissue), to sustain the tissue in a condition where its cells remain viable and metabolically active for a span of hours or days after the animal was killed. Compared to ocular tissue samples from mice or rats, ocular tissues from animals such as cows or pigs are much easier to handle and work with, and they also provide more relevant results, if dimensional factors are important (such as, for example, when the permeation of a drug or nutrient into or through lens tissue is important).

6. Primate Tests

Primates include lemurs, monkeys, and apes. While they are expensive to raise, keep, and test, they nevertheless provide animal models that, in some situations, will provide better and more applicable and relevant data than any other type of animal test, short of a human clinical trial. Therefore, they must be kept in mind as one option. In many situations, to keep costs under control, it may be possible to “piggyback” a vision-related test on top of some other type of ongoing test (such as a cancer-related test), using the same animals that are being tested for other purposes.

Clcinical Trials and Meta-Trials

The best data regarding the efficacy of macular protection formulations can be generated by tests on humans, since millions of people are suffering from or at elevated risk of the disease, and they will eagerly cooperate in any realistic and reasonable effort to help preserve their eyesight. Painless and noninvasive analytical methods can be used, including: (1) vision acuity tests, such as Amsler grids, charts having rows of progressively smaller letters or numbers, etc.; (2) methods for measuring macular pigment density, such as flicker photometry, spectral fundus reflectometry, and scanning laser ophthalmoscopy; and (3) methods for measuring the quantities and average diameters of drusen and/or lipofuscin, two types of debris found in the retinas of people suffering from macular degeneration.

It should also be noted that a condition referred to as “geographic atrophy” provides a useful indicator of the progression of macular degeneration. Briefly, “geographic atrophy” is said to occur when retinal lesions reach a point where they begin to show relatively clear and distinct areas and sizes (often called patches), which can be seen in certain types of retinal photographs. When this stage of the disease is reached, it usually indicates that the patient has passed beyond a long entry stage involving slow and gradual deterioration over a span of years, and has entered a phase of accelerated and rapid deterioration that will cause the lesions to grow significantly larger, over the next weeks and months. Therefore, the ability of a candidate treatment to slow down the spread of geographic lesions, once those have started to form, can provide useful data within a few months, rather than requiring years of monitoring.

These types of indicators and trials can be carried out at schools of medicine or optometry, using conventional methods. However, it should also be recognized that there is a potential way to gather useful and statistically reliable data based on large numbers of test and control subjects, without waiting for years for the results of a large privately-funded or government-sponsored multi-center trial to slowly emerge. This approach involves “meta-trials” that can involve dozens or even hundreds of optometrists and/or ophthalmologists across the nation (especially in southern locations with high levels of sun exposure and large numbers of retirees, such as Florida or Arizona). Each participating optometrist or ophthalmologist can go through one or more orientation and/or training sessions to ensure that they gather data in a consistent manner. Photographs of patient retinas can be taken at designated centers that have the necessary equipment (including schools of optometry, and the offices of participating ophthalmologists), and the resulting photographs can be submitted to a single scoring location, where they will be assigned random numbers, and evaluated by trained analysts who do not know what treatment any number-coded patient received.

By using this approach, even though each participating optometrist or ophthalmologist will be working with only a limited number of patients, all of the data they will gather will be submitted to, and incorporated into, a larger pool of data, which can rapidly add up to thousands or even tens of thousands of patients for each treatment group. Since thousands of macular degeneration patients would readily and willingly volunteer for such trials, since thousands of optometrists and ophthalmologists already have the skills necessary to prescribe and monitor out such treatments, and since retinal photography equipment is available at dozens or even hundreds of locations across America, meta-trials should be regarded as a minimum-cost and rapid way to gather reliable statistical data on candidate multiple-agent formulations as disclosed herein, for preventing or treating macular degeneration.

Animal Tests and Human Trials on Brain Protection

Protection of the brain against Alzheimer-related and neurotoxic risks, posed by high dosages of zinc, is a crucial part of this invention. Therefore, attention must be given to animal models and human tests that can be used to evaluate the efficacy of various components of the multi-component formulations described herein.

With regard to animal models of β-amyloid plaques and Alzheimer's disease, mice, rats, and other rodents do not normally create β-amyloid plaques in their brains. However, genetically engineered strains of mice have been created that do accumulate β-amyloid plaques. These strains of mice, and tests that have been carried out using such mice, are described in articles such as Phinney et al 2003 and Higgins et al 2003.

Dogs offer better models than mice for certain types of studies on aging, senility, and Alzheimer's disease. Among other advantages, dogs naturally create and accumulate β-amyloid plaques in their brains, and many aged dogs suffer from behaviorally-indicated forms of senility that have apparent correlations with human senility, concerning dietary and blood-borne antioxidants and other compounds, brain similarities following autopsies, quantifiable learning and memory impairments, and other factors. These factors are described in articles such as Head et al 2000 and 2002, Cotman et al 2002, Dimakopoulos et al 2002, Rofina et al 2003, and Torp et al 2003.

Since aging dogs offer better models for studying Alzheimer's disease and senility than mice or rats, the inventors herein propose the establishment of a coordinated network of “nursing homes” for severely aged dogs who have become incontinent, senile, or are otherwise approaching the natural ends of their lives. Such “canine nursing homes” can be staffed and operated by skilled supervisors and research managers, while much of the daily care and monitoring of prescribed diets and medicines can be handled by people who enjoy the companionship of animals, and who suffer from Down's syndrome or other mental or developmental disabilities that would not prevent them from being good companions and caretakers for aging dogs. Alternately or additionally, keeping and caring properly for aged dogs could be structured in a way that could offer good educational and/or behavioral therapy for troubled adolescents, children in foster homes, prisoners who are undergoing a halfway-house or paroled transition back into society, and various other people in similar conditions.

Accordingly, “canine nursing homes” could provide humane and benevolent alternatives to euthanasia, for people who can no longer keep a family pet that has become incontinent. If they are properly managed, they could offer well-controlled and completely painless methods for testing various types of dietary and nutritional supplements, and Alzheimer-preventive test drugs, which could be fed in controlled manners to different populations of dogs. Accordingly, this approach to creating coordinated and interacting networks of “canine nursing homes” is being proposed herein, as a method for accelerating and improving the testing of nutritional supplements, and drugs that are being tested for preventing or treating macular degeneration, Alzheimer's disease, or other neurodegenerative diseases.

On the subject of human clinical trials for testing nutritional supplements or candidate drugs that may be able to prevent or slow the progession of Alzheimer's disease, it should be recognized that major strides have been made within the past decade, in the field of painless and non-invasive imaging of β-amyloid plaques, and levels of brain activity, in human brains. This type of imaging, using methods such as positron emission tomography or magnetic resonance imaging, can be enhanced by certain types of “markers” that will bind to amyloid plaques. These methods and reagents are described in articles such as Klunk et al 2003 and Heckl et al 2004.

Accordingly, the animal models and human tests described above, and other testing methods and approaches known to those skilled in the art, can be used to evaluate the various candidate macular protective and brain-protective agents disclosed herein, alone and/or in various combinations.

Thus, there has been shown and described improved formulations that can perform more effectively than AREDS-1 formulations in preventing and treating macular degeneration, while at the same time reducing the neurotoxic risks that are posed by the exceptionally heavy dosages of zinc contained in AREDS-1 formulations. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

### REFERENCES

Alagoz G, et al, “L-carnitine in experimental retinal ischemia-reperfusion injury,” Ophthalmologica 2002 March-April; 216(2): 144-50
Ambati, J., et al, “An animal model of age-related macular degeneration in senescent Ccl-2 or Ccr-2-deficient mice,” Nat Med 9: 1390-97 (2003)
Ambati, J., et al, “Age-related macular degeneration: etiology, pathogenesis, and therapeutic strategies,” Surv Ophthamol 48: 257-93 (2003)
Areias F M, et al, “Antioxidant effect of flavonoids after ascorbate/Fe(2+)-induced oxidative stress in cultured retinal cells,” Biochem Pharmacol 2001 Jul. 1; 62(1): 111-8
Armstrong, D., et al, “Lipid hydroperoxide stimulates retinal neovascularization in rabbit retina through expression of tumor necrosis factor-alpha, vascular endothelial growth factor and platelet-derived growth factor,” Angiogenesis 2(1): 93-104 (1998)
Babizhayev M A, et al, “Efficacy of N-acetylcarnosine in the treatment of cataracts,” Drugs R D. 2002; 3(2):87-103
Barnham K J, et al, “Neurotoxic, redox-competent Alzheimer's beta-amyloid is released from lipid membrane by methionine oxidation,” J Biol. Chem. 2003; 278: 42959-65
Beaumont P, “Zinc and macular degeneration,” Arch Ophthalmol 111: 1023 (1993)
Beecher G R, “Phytonutrients' role in metabolism: effects on resistance to degenerative processes,” Nutr Rev. 1999 September; 57(9 Pt 2):S3-6
Beecher G R, “Overview of dietary flavonoids: nomenclature, occurrence and intake,” J Nutr. 2003 October; 133(10):3248S-3254S
Berr, C, et al, “Cognitive decline is associated with systemic oxidative stress,” J Amer Geriatr Soc 2000; 48: 1285-91
Blodi B A, “Nutritional supplements in the prevention of age-related macular degeneration,” Insight 2004 January-March; 29(1): 15-6
Borenshtein D, et al, “Cataract development in diabetic sand rats treated with alpha-lipoic acid and its gamma-linolenic acid conjugate,” Diabetes Metab Res Rev 2001 January-February; 17(1): 44-50
Brakenhielm E, et al, “Suppression of angiogenesis, tumor growth, and wound healing by resveratrol, a natural compound in red wine and grapes,” FASEB J. 2001 August; 15(10): 1798-800
Burton, G W, et al, “Beta-carotene: An unusual type of lipid antioxidant,” Science 224: 569-573 (1984)
Bush A I, et al, “Rapid induction of Alzheimer Abeta amyloid formation by zinc,” Science. 1994 Sep. 2; 265(5177): 1464-7
Bush A I, “Metal complexing agents as therapies for Alzheimer's disease,” Neurobiol Aging. 2002 November-December; 23(6): 1031-8.
Byrne S, et al, “Current concepts and recent advances in the management of age-related macular degeneration,” Ir J Med Sci 2003 October-December; 172(4): 185-90
Canzoniero L M, et al, “Membrane-permeant chelators can attenuate Zn2+-induced cortical neuronal death,” Neuropharmacology. 2003 September; 45(3): 420-8.
Cao Y, et al, “Antiangiogenic-mechanisms of diet-derived polyphenols,” J Nutr Biochem 2002 July; 13(7): 380-390
Carpenter, K. L., et al, “The carotenoids beta-carotene, canthaxanthin and zeaxanthin inhibit macrophage-mediated LDL oxidation,” FEBS Lett 401(2-3): 262-6 (1997)
Castillo M, et al, “Effects of hypoxia on retinal pigmented epithelium cells: protection by antioxidants,” Ophthalmic Res 2002 November-December; 34(6): 338-42
Chaney M O, et al, “Abeta, aging, and Alzheimer's disease: a tale, models, and hypotheses,” Neurol Res. 2003 September; 25(6): 581-9
Chen F, et al, “An experimental research of taurine on H₂O₂-induced bovine lens epithelial cell apoptosis,” Zhonghua Yan Ke Za Zhi. 2000 July; 36(4):272-4, 17
Cherny R A, et al, “Aqueous dissolution of Alzheimer's disease Abeta amyloid deposits by biometal depletion,” J Biol. Chem. 1999 Aug. 13; 274(33): 23223-8
Cherny R A, et al, “Treatment with a copper-zinc chelator markedly and rapidly inhibits beta-amyloid accumulation in Alzheimer's disease transgenic mice,” Neuron. 2001 June; 30(3): 665-76
Chew, E Y, et al, “Cognitive Function in the Age-Related Eye Disease Study (AREDS),” Invest. Ophthalmol. Vis. Sci. 2004 45: E-Abstract 3056, www.arvo.com
Chew, B. P., et al, “Dietary lutein inhibits mouse mammary tumor growth by regulating angiogenesis and apoptosis,” Anticancer Res 23(4): 3333-9 (2003)
Chidlow G, et al, “Alpha-lipoic acid protects the retina against ischemia-reperfusion,” Neuropharmacology. 2002 November; 43(6):1015-25
Choi D W, et al, “Zinc and brain injury,” Annu Rev Neurosci. 1998; 21: 347-75.
Connor W E., “Importance of omega-3 fatty acids in health and disease,” Am J Clin Nutr. 2000 January; 71(1 Suppl):171S-5S
Cotman C W, et al, “Brain aging in the canine: a diet enriched in antioxidants reduces cognitive dysfunction,” Neurobiol Aging 2002 September-October; 23(5): 809-18
Cousins R J, “Systemic transport of zinc,” pp. 78-93 in Mills, ed., Zinc in Human Biology (Springer-Verlag, 1989)
Craft N E, et al, “Carotenoid, tocopherol, and retinol concentrations in elderly human brain,” J Nutr Health Aging. 2004; 8(3): 156-62
Cuajungco M P, et al, “Metal chelation as a potential therapy for Alzheimer's disease,” Ann N Y Acad. Sci. 2000; 920: 292-304.
Cuajungco M P, et al, “Zinc takes the center stage: its paradoxical role in Alzheimer's disease,” Brain Res Brain Res Rev. 2003 January; 41(1): 44-56.
D'Amato, R, et al, Macular Degeneration: The Latest Scientific Discoveries and Treatments for Preserving Your Sight (Walker & Company, 2000)
Devamanoharan P S, et al, “Oxidative stress to rat lens in vitro: protection by taurine,” Free Radic Res 1998 September; 29(3): 189-95
DiLeo M A, et al, “Potential therapeutic effect of antioxidants in experimental diabetic retina: a comparison between chronic taurine and vitamin E plus selenium supplementations,” Free Radic Res 2003 March; 37(3): 323-30
Dimakopoulos A C, et al, “Aspects of neurodegeneration in the canine brain,” J Nutr 2002 June; 132(6 Suppl 2): 1579S-82S
Dithmar, S., et al, “Murine high-fat diet and laser photochemical model of basal deposits in Bruch membrane,” Arch Opthamol 119: 1643-9 (2001)
Dithmar, S., et al, “Ultrastructural changes in Bruch's membrane of apolipoprotein E-deficient mice,” Invest Opthalmol Vis Sci 41: 2035-42 (2000)
Erlund I, et al, “Consumption of black currants, lingonberries and bilberries increases serum quercetin concentrations,” Eur J Clin Nutr. 2003 January; 57(1):37-42
Feher J, et al, “Mitotropic compounds for the treatment of age-related macular degeneration. The metabolic approach and a pilot study,” Ophthalmologica. 2003 September-October; 217(5):351-7
Finefrock A E, et al, “Current status of metals as therapeutic targets in Alzheimer's disease,” J Am Geriatr Soc. 2003 August; 51(8): 1143-8.
Flinn J M, et al, “Enhanced zinc consumption causes memory deficits and increased brain levels of zinc,” Physiol Behav. 2005 Jan. 17; 83(5): 793-803
Ford, E. S., et al, “C-reactive protein concentration and concentrations of blood vitamins, carotenoids, and selenium among United States adults,” Eur J Clin Nutr 57(9): 1157-63 (2003)
Frederickson C J, Bush A I, “Synaptically released zinc: physiological functions and pathological effects,” Biometals. 2001 September-December; 14(3-4): 353-66.
Friedlich A L, et al, “Neuronal zinc exchange with the blood vessel wall promotes cerebral amyloid angiopathy in an animal model of Alzheimer's disease, J. Neurosci. 2004 Mar. 31; 24(13): 3453-9
Gao X, et al, “Powerful and prolonged protection of human retinal pigment epithelial cells, keratinocytes, and mouse leukemia cells against oxidative damage: the indirect antioxidant effects of sulforaphane,” PNAS USA 2001; 98: 15221-6
Gaynes B I, “AREDS misses on safety,” Arch Ophthalmol. 2003 March; 121(3): 416-7
Gonzalez, S., et al, “Dietary lutein/zeaxanthin decreases ultravoilet B-induced epidermal hyperproliferation and acute inflammation in hairless mice,” J Invest Dermatol 121(2): 399-405 (2003)
Goralska M, et al, “Alpha lipoic acid changes iron uptake and storage in lens epithelial cells,” Exp Eye Res 2003 February; 76(2): 241-8
Hawkins W R, “Zinc supplementation for macular degeneration,” Arch Ophthalmol 109: 1345 (1991)
Head E, et al, “Initiation and propagation of molecular cascades in human brain aging: insight from the canine model to promote successful aging,” Prog Neuropsychopharmacol Biol Psychiatry 2000 July; 24(5): 777-86
Head E, et al, “Insights into Abeta and presenilin from a canine model of human brain aging,” Neurobiol Dis. 2002 February; 9(1): 1-10
Heckl S, “Molecular imaging: Bridging the gap between neuroradiology and neurohistology,” Histol Histopathol 2004 April; 19(2): 651-68
Higgins G A, et al, “Transgenic mouse models of Alzheimer's disease: phenotype and application,” Behav Pharmacol. 2003 September; 14(5-6): 419-38
Hipkiss A R, et al, “Pluripotent protective effects of carnosine, a naturally occurring dipeptide,” Ann N Y Acad. Sci. 1998 Nov. 20; 854:37-53
Jampol L M, “Antioxidants, zinc, and age-related macular degeneration: results and recommendations,” Arch Ophthalmol. 2001 October; 119(10): 1533-4
Jampol L M, “AREDS—two years later,” Arch Ophthalmol. 2003 November; 121(11): 1634-6
Jeffrey B G, et al, “The role of docosahexaenoic acid in retinal function,” Lipids 2001 September; 36(9): 859-71
Joussen A M, et al, “Treatment of corneal neovascularization with dietary isoflavonoids and flavonoids,” Exp Eye Res 2000 November; 71(5): 483-7
Jung S H, et al, “Isoflavonoids from the rhizomes of Belamcanda chinensis and their effects on aldose reductase and sorbitol accumulation in streptozotocin induced diabetic rat tissues,” Arch Pharm Res 2002 June; 25(3): 306-12
Kagan B L, et al, “The channel hypothesis of Alzheimer's disease: current status,” Peptides. 2002 July; 23(7): 1311-5.
Kahkonen M P, et al, “Berry phenolics and their antioxidant activity,” J Agric Food Chem. 2001 August; 49(8):4076-82
Kahkonen M P, et al, “Berry phenolics and their antioxidant activity,” J Agric Food Chem 2001 August; 49(8): 4076-82
Kar S, Quirion R, “Amyloid beta peptides and central cholinergic neurons: functional interrelationship and relevance to Alzheimer's disease pathology,” Prog Brain Res. 2004; 145: 261-74.
Khachik F, et al, “Transformations of selected carotenoids in plasma, liver, and ocular tisues of humans and in nonprimate animal models,” Invest Ophthalmol Vis Sci 2002; 43(11): 3383-3392
Khan M, et al, “S-Nitrosoglutathione reduces inflammation and protects brain against focal cerebral ischemia in a rat model of experimental stroke,” J Cereb Blood Flow Metab. 2005; 25: 177-92.
Kilic F, et al, “Modeling cortical cataractogenesis: XX. In vitro effect of alpha-lipoic acid on glutathione concentrations in lens in model diabetic cataractogenesis,” Biochem Mol Biol Int 1998 October; 46(3): 585-95
Klunk W E, et al, “Imaging the pathology of Alzheimer's disease: amyloid-imaging with positron emission tomography,” Neuroimaging Clin N Am 2003 November; 13(4): 781-9, ix
Kocer I, et al, “Protection of the retina from ischemia-reperfusion injury by L-carnitine in guinea pigs,” Eur J Ophthalmol. 2003 January-February; 13(1):80-5
Konwinski R R, et al, “Oltipraz, 3H-1,2-dithiole-3-thione, and sulforaphane induce overlapping and protective antioxidant responses in murine microglial cells,” Toxicol Lett. 2004; 153: 343-55.
Kowluru R A, et al, “Diabetes-induced mitochondrial dysfunction in the retina,” Invest Ophthalmol Vis Sci. 2003 December; 44(12):5327-34
Kowluru R A, “Diabetes-induced elevations in retinal oxidative stress, protein kinase C and nitric oxide are interrelated,” Acta Diabetol. 2001 December; 38(4): 179-85
Kraft A D, et al, “Nuclear factor E2-related factor 2-dependent antioxidant response element activation by tert-butylhydroquinone and sulforaphane occurring preferentially in astrocytes conditions neurons against oxidative insult,” J. Neurosci. 2004; 24:1101-12
Lee J M, et al, “Zinc translocation accelerates infarction after mild transient focal ischemia,” Neuroscience. 2002; 115(3): 871-8.
Lee, S J, et al, “Axtaxanthin inhibits nitric oxide production and inflammatory gene expression by suppressing I(kappa)B kinase-dependent NF-kappaB activation,” Mol Cells 16(1): 97-105 (2003)
Lee, E H, et al, “Dietary lutein reduces UV radiation-induced inflammation and immunosuppression,” J Invest Dermatol 122(5): 510-7 (2004)
Leitzmann M F, et al, “Zinc supplement use and risk of prostate cancer,” J Natl Cancer Inst. 2003 Jul. 2; 95(13): 1004-7
Lim, J I, editor, Age-Related Macular Degeneration (Marcel Dekker, 2002) Lobner D, et al “Zinc-induced neuronal death in cortical neurons,” Cell Mol Biol (Noisy-le-grand). 2000 June; 46(4): 797-806.
Lorenz P, et al, “Oxyresveratrol and resveratrol are potent antioxidants and free radical scavengers: effect on nitrosative and oxidative stress derived from microglial cells,” Nitric Oxide. 2003 September; 9(2):64-76
Maichuk I F, et al, “[Development of carnosine eyedrops and assessing their efficacy in corneal diseases],” Vestn Oftalmol 1997 November-December; 113(6): 27-31
Maitra I, et al, “Stereospecific effects of R-lipoic acid on buthionine sulfoximine-induced cataract formation in newborn rats,” Biochem Biophys Res Commun. 1996 Apr. 16; 221(2):422-9
Manzanas L, et al, “Oral flavonoids, chromocarb diethylamine salt and cyaninosides chloride, to eliminate lipoperoxidation postvitrectomy,” Exp Eye Res 2002 January; 74(1): 23-8
Manzerra P, et al, “Zinc induces a Src family kinase-mediated up-regulation of NMDA receptor activity and excitotoxicity,” Proc Natl Acad Sci USA. 2001 Sep. 25; 98(20): 11055-61.
Maoka T, et al, “The first isolation of enantiomeric and meso-zeaxanthin in nature,” Comp Biochem Physiol (B) 1986; 83: 121-124
Matsuda H, et al, “Structural requirements of flavonoids and related compounds for aldose reductase inhibitory activity,” Chem Pharm Bull (Tokyo). 2002 June; 50(6):788-95
Miceli, M. V., et al, “Pathologic changes in the retinal pigment epithelium and Bruch's membrane of fat-fed atherogenic mice,” Curr Eye Res 20: 8-16 (2000)
Militante J D, et al, “Taurine: evidence of physiological function in the retina,” Nutr Neurosci. 2002 April; 5(2):75-90
Morimitsu Y, et al, “A sulforaphane analogue that potently activates the Nrf2-dependent detoxification pathway,” J Biol. Chem. 2002; 277:3456-63
Moskovitz J, “Roles of methionine suldfoxide reductases in antioxidant defense, protein regulation and survival,” Curr Pharm Des. 2005; 11: 1451-7.
Moyad M A, “Zinc for prostate disease and other conditions: a little evidence, a lot of hype, and a significant potential problem,” Urol Nurs. 2004 February; 24(1): 49-52
Murayama K, et al, “Fish oil (polyunsaturated fatty acid) prevents ischemic-induced injury in the mammalian retina,” Exp Eye Res 2002 June; 74(6): 671-6
Newsome, D A, et al, “Oral zinc in macular degeneration,” Arch Ophthalmol 106: 192-198 (1988)
Obrosova I G, et al, “Taurine counteracts oxidative stress and nerve growth factor deficit in early experimental diabetic neuropathy,” Exp Neurol. 2001 November; 172(1):211-9
Obrosova I, et al, “Diabetes-induced changes in lens antioxidant status, glucose utilization and energy metabolism: effect of DL-alpha-lipoic acid,” Diabetologia. 1998 December; 41(12): 1442-50
Obrosova I G, et al, “Early changes in lipid peroxidation and antioxidative defense in diabetic rat retina: effect of DL-alpha-lipoic acid,” Eur J. Pharmacol. 2000 Jun. 9; 398(1): 139-46
Obrosova I G, et al, “Effect of dietary taurine supplementation on GSH and NAD(P)-redox status, lipid peroxidation, and energy metabolism in diabetic precataractous lens,” Invest Ophthalmol Vis Sci. 1999 March; 40(3):680-8
Ohgami, K., et al, “Effects of astaxanthin on lipopolysaccharide-induced inflammation in vitro and in vivo,” Invest Ophthamol Vis Sci 44(6): 2694-701 (2003)
Okuyama H, et al, “Alpha-linolenate-deficiency-induced alterations in brightness discrimination learning behavior and retinal function in rats,” World Rev Nutr Diet 2001; 88: 35-40
Packer L, “Antioxidant properties of lipoic acid and its therapeutic effects in prevention of diabetes complications and cataracts,” Ann N Y Acad Sci 1994 Nov. 17; 738: 257-64
Pasantes-Morales H, et al, “Treatment with taurine, diltiazem, and vitamin E retards the progressive visual field reduction in retinitis pigmentosa: a 3-year follow-up study,” Metab Brain Dis 2002 September; 17(3): 183-97
Peluso G, et al, “Carnitine protects the molecular chaperone activity of lens alpha-crystallin and decreases the post-translational protein modifications induced by oxidative stress,” FASEB J. 2001 July; 15(9):1604-6
Pessotto P, et al, “In experimental diabetes the decrease in the eye of lens carnitine levels is an early important and selective event,” Exp Eye Res. 1997 February; 64(2):195-201
Peto, T., et al, “Zinc in drusen,” Invest. Ophthalmol. Vis. Sci. 2005 46: E-Abstract 3008, www.arvo.com
Phinney A L, et al, “Mouse models of Alzheimer's disease: the long and filamentous road,” Neurol Res. 2003 September; 25(6): 590-600
Polit L, et al, “Effects of docosahexaenoic acid on retinal development: cellular and molecular aspects,” Lipids. 2001 September; 36(9):927-35
Pulido J S, Blake C R, “Special considerations in the guidelines for high-dose vitamin supplementation in patients with age-related macular degeneration,” Arch Ophthalmol. 2004 April; 122(4): 662
Rinaldi, P. et al, “Plasma antioxidants are similarly depleted in mild cognitive impairment and in Alzheimer's disease,” Neurobiol of Aging 2003 24: 915-919
Ritchie C W, et al, “Metal-protein attenuation with iodochlorhydroxyquin (clioquinol) targeting Abeta amyloid deposition and toxicity in Alzheimer disease: a pilot phase 2 clinical trial,” Arch Neurol. 2003 December; 60(12): 1685-91.
Robert A M, et al, “[Protection of cornea against proteolytic damage. Experimental study of procyanidolic oligomers (PCO) on bovine cornea] J Fr Ophtalmol. 2002 April; 25(4):351-5
Rofina J, et al, “Canine counterpart of senile dementia of the Alzheimer type: amyloid plaques near capillaries but lack of spatial relationship with activated microglia and macrophages,” Amyloid 2003 June; 10(2): 86-96
Rotstein N P, et al, “Protective effect of docosahexaenoic acid on oxidative stress-induced apoptosis of retina photoreceptors,” Invest Ophthalmol Vis Sci 2003 May; 44(5): 2252-9
Rulon L L, et al, “Serum zinc levels and Alzheimer's disease,” Biol Trace Elem Res. 2000 Summer; 75(1-3): 79-85.
Ryu R, et al, “Depletion of intracellular glutathione mediates zinc-induced cell death in rat primary astrocytes,” Exp Brain Res. 2002 March; 143(2): 257-63.
Sadhu, C. and Gedamu, L., “Metal-specific post-transcriptional control of human metallothionein genes,” Mol. Cell. Biol. 9: 5738-41 (1989)
Sheline C T, et al, “Involvement of poly ADP ribosyl polymerase-1 in acute but not chronic zinc toxicity,” Eur J. Neurosci. 2003 September; 18(6): 1402-9.
Siegel, D., “AREDS investigators distort findings,” Arch Ophthalm 2002 January; 120(1): 100-101.
Sparrow J R, et al, “A2E-epoxides damage DNA in retinal pigment epithelial cells: Vitamin E and other antioxidants inhibit A2E-epoxide formation,” J Biol Chem 2003 May 16; 278(20): 18207-13
Stadtman E R, et al, “Oxidation of methionine residues of proteins: biological consequences,” Antioxid Redox Signal 2003; 5: 577-82.
Stadtman E R, et al, “Methionine oxidation and aging,” Biochim Biophys Acta. 2005; 1703: 135-40
Stoyanovsky D A, et al, “Endogenous ascorbate regenerates vitamin E in the retina directly and in combination with exogenous dihydrolipoic acid,” Curr Eye Res 1995 March; 14(3): 181-9
Suh, S W, et al, “Evidence that synaptically released zinc contributes to neuronal injury after traumatic brain injury” Brain Research 2000 852:I 268-273
Tanito M, et al, “Sulforaphane induces thioredoxin through the antioxidant-responsive element and attenuates retinal light damage in mice,” Invest Ophthalmol Vis Sci. 2005; 46:979-87
Thomson L R, et al, “Elevated retinal zeaxanthin and prevention of light-induced photoreceptor cell death in quail,” Invest Ophthalmol Vis Sci 2002 November; 43(11): 3538-49
Torp R, et al, “Identification of neuronal plasma membrane microdomains that colocalize beta-amyloid and presenilin: implications for beta-amyloid precursor protein processing,” Neuroscience 2003; 120(2): 291-300
Trempe C L, “Zinc and macular degeneration,” Arch Ophthalmol 110: 1517 (1992)
Turner P R, et al, “Roles of amyloid precursor protein and its fragments in regulating neural activity, plasticity and memory,” Prog Neurobiol. 2003 May; 70(1): 1-32
Vallee, B I, et al, “The biochemical basis of zinc physiology,” Physiological Reviews 73: 79-118 (1993)
Vallee, B I, “Zinc: Biochemistry, toxicology, and clinical pathology,” Biofactors 1: 31-36 (1988)
vanHerpen-Broekmans, W. M., et al, “Serum carotenoids and vitamins in relation to markers of endothelial function and inflammation,” Eur J Epidemiol 19(10): 915-21 (2004)
Walrand, S., et al, “In vivo and in vitro evidences that carotenoids could modulate the neutrophil respiratory burst during dietary manipulation,” Eur J Nutr May 6: 1-7 (2004)
Yaffe K, et al, “Impact of antioxidants, zinc, and copper on cognition in the elderly: a randomized, controlled trial,” Neurology. 2004 Nov. 9; 63(9):1705-7
Yamakoshi J, et al, “Procyanidin-rich extract from grape seeds prevents cataract formation in hereditary cataractous (ICR/f) rats,” J Agric Food Chem 2002 Aug. 14; 50(17): 4983-8
Yermolaieva 0, et al, “Methionine sulfoxide reductase A protects neuronal cells against brief hypoxia/reoxygenation,” PNAS USA 2004; 101: 1159-64
Yoon H S, et al, “Genistein produces reduction in growth and induces apoptosis of rat RPE-J cells,” Curr Eye Res. 2000 March; 20(3):215-24
Yuzbasiyan, G V, et al, “The therapeutic use of zinc in macular degeneration,” Arch Ophthalmol 107: 1723-24 (1989)
Zarbin M A, “Current concepts in the pathogenesis of age-related macular degeneration,” Arch Ophthalmol 2004 April; 122(4): 598-614

1. A nutrient formulation for preventing or treating at least one ocular disorder in mammals, wherein said nutrient formulation comprises:
a. zeaxanthin, in a daily dosage of at least about 3 milligrams;
b. elemental zinc, in a daily dosage of about 15 to about 40 milligrams,
wherein said nutrient formulation is also characterized by an absence of beta-carotene at a daily dosage higher than twice the recommended daily allowance.

2. A nutrient formulation of claim 1, comprising zeaxanthin in a daily dosage of at least about 5 milligrams.

3. A nutrient formulation of claim 1, comprising zeaxanthin in a daily dosage of at least about 10 milligrams.

4. A nutrient formulation of claim 1, comprising elemental zinc in a daily dosage of 30 to 40 milligrams.

5. A nutrient formulation of claim 1, which also contains at least one additional neuroprotective agent selected from the group consisting of mitochondrial boosting compounds, glutathioone boosting compounds, carnosine, agents that stimulate methionine sulfoxide reductase enzymes, and plant-derived flavonoids.

6. A nutrient formulation of claim 1, which also contains Vitamin C and Vitamin E.

7. The nutrient formulation of claim 1, which is in a unit dosage form.

8. The nutrient formulation of claim 7, wherein the unit dosage form is selected from the group consisting of capsules, tablets, liquids accompanied by instructions for consumers to ingest a measured volume of liquid each day, and powdered or granular formulations accompanied by instructions for consumers to ingest a measured volume of powdered or granular formulation each day.

9. The nutrient formulation of claim 1, wherein the nutrient formulation has been added to and is contained within a food or beverage material.

10. A method of manufacturing a nutritional supplement for preventing or treating ocular disorders, comprising the step of incorporating into said nutritional supplement:
a. zeaxanthin, in a daily dosage of at least about 3 milligrams;
b. elemental zinc, in a daily dosage of about 15 to about 40 milligrams,
wherein said nutrient formulation is also characterized by an absence of beta-carotene at a daily dosage higher than twice the recommended daily allowance.

11. The method of claim 10, wherein said nutrient formulation is in a unit dosage form.
